(19) United States
(12) Patent Application Publication
(10) Pub. No.: US 2022/0404353 A1
(43) Pub. Date: Dec. 22, 2022

(54) SYSTEMS AND DEVICES FOR SAMPLE PREPARATION AND ANALYTE DETECTION

(71) Applicant: Juno Diagnostics, Inc., San Diego, CA (US)

(72) Inventors: Krisna C. Bhargava, San Diego, CA (US); Jim Chauvapun, San Diego, CA (US); Mathias Ehrich, San Diego, CA (US); Megan Monier, Corona, CA (US); Michael Van Nguyen, San Diego, CA (US); Dirk van den Boom, Encinitas, CA (US)

(21) Appl. No.: 17/780,227

(22) PCT Filed: Nov. 20, 2020
(86) PCT No.: PCT/US2020/061599

§ 371 Date: May 26, 2022

Related U.S. Application Data
(60) Provisional application No. 62/941,329, filed on Nov. 27, 2019.

(51) Int. Cl. G01N 33/543; G01N 33/53; C12Q 1/6804; C12N 15/10

(52) U.S. Cl. CPC G01N 33/54388; G01N 33/5308; C12Q 1/6804; C12N 15/101

(57) ABSTRACT

Provided are systems and methods of sample preparation and analyte detection.

### CROSS-REFERENCE

This application claims the benefit of U.S. Provisional Application No. 62/941,329, filed Nov. 27, 2019, which application is incorporated herein by reference in its entirety.

### SUMMARY

Aspects disclosed herein provide devices for preparing a sample for analyte detection comprising: processor comprising: a filter configured to filter solid particulate from the sample, and an enricher downstream from the filter and configured to increase a quantity of target analytes in the sample; a fluid supply comprising a reagent and a pump that move the reagents from the fluid supply and move the sample to the enricher; and a fluid routing network comprised of fluid pathway and valve to direct flow of the sample and the reagents to the enricher. In some embodiments, the devices further comprises an electronics and software subsystem that controls the pump and the valve. In some embodiments, the valve is a rotary valve. In some embodiments, the device further comprises an electronics and software subsystem that controls the pump and the rotary valve. In some embodiments, the processor further comprises a washer downstream from the filter and upstream from the enricher and configured to separate the target analytes from other substances within the sample. In some embodiments, the processor further comprises a hybridizer downstream from the filter and upstream from the enricher, the hybridizer configured to bind the target analytes to one or more antibodies of high affinity. In some embodiments, the processor further comprises an eluter downstream from the hybridizer and upstream from the enricher, the eluter is configured to isolate analytes to be detected from the sample in an eluate. In some embodiments, the processor further comprises a diluter downstream from the enricher, the diluter configured to dilute the eluate in an aqueous buffer. In some embodiments, the processor further comprises a detector downstream from all components of the processor, the detector configured to detect the target analytes in the eluate. In some embodiments, the detector produces an optically detectable signal. In some embodiments, the detector comprises a chromatography device. In some embodiments, the chromatography device is a lateral flow assay. In some embodiments, at least one of the hybridizer, the eluter, the enricher, or the diluter comprise an air vent. In some embodiments, the processor further comprises a diluter downstream from the enricher, the diluter configured to dilute the sample in an aqueous buffer. In some embodiments, the processor further comprises a detector downstream from all components of the processor, the detector configured to detect the target analytes in the sample. In some embodiments, the sample has a volume comprising at most or about 400 microliters (μl), 350 μl, 300 μl. 250 μl, 200 μl, 150 μl, 100 μl, 50 μl, 45 μl, 40 μl, 35 μl, 30 μl or less. In some embodiments, the sample is whole blood. In some embodiments, the target analytes comprise a target region of cell-free deoxyribonucleic acid (DNA). In some embodiments, the cell-free DNA is fragmented. In some embodiments, the sample comprises an amount of the target analytes comprising between or about 4pg to 100pg, 4pg to 150pg, 4pg to 200pg, 4pg to 250pg, 4pg to 300pg, 4pg to 350pg, 4pg to 400pg, 4pg to 450pg, 4pg to 500pg, 10pg to 100pg, 10pg to 150pg, 10pg to 200pg, 10pg to 250pg, 10pg to 300pg, 10pg to 350pg, 10pg to 400pg, 10pg to 450pg, 10pg to 500pg, 20pg to 100pg, 20pg to 150pg, 20pg to 200pg, 20pg to 250pg, 20pg to 300pg, 20pg to 350pg, 20pg to 400pg, 20pg to 450pg, 20pg to 500pg, 30pg to 100pg, 30pg to 150pg, 30pg to 200pg, 30pg to 250pg, 30pg to 300pg, 30pg to 350pg, 30pg to 400pg, 30pg to 450pg, or 30pg to 500pg.

Aspects disclosed herein provide systems comprising: one or more devices of the devices described above; at least one controller for controlling the one or more devices; and at least one interface for manipulating the at least one controller. In some embodiments, the system further comprises a sample collector configured to obtain the sample from a subject. In some embodiments, the sample collector is operably coupled to a transdermal puncture device. In some embodiments, the transdermal puncture device comprises a microneedle, microneedle array, or microneedle patch.

Aspects disclosed herein provide methods for preparing a sample and for analyte detection using any of the embodiments of the device disclosed above, the method comprising: receiving the sample comprising the target analytes at an inlet of the filter; filtering the sample with the filter, thereby producing a filtered sample; mixing the filtered sample with the aqueous solution in the hybridizer; hybridizing the filtered sample mixed with the aqueous solution in the hybridizer, thereby producing the hybridized solution; mixing the hybridized solution with a solvent in the eluter, thereby producing the eluate; mixing the eluate with an enrichment solution in the enricher; enriching the eluate mixed with the enrichment solution in the enricher, thereby producing an enriched sample; diluting the enriched sample with an aqueous buffer in the diluter, thereby producing a diluted sample; introducing the diluted sample to the detector to create at least one optically detectable signal; and producing an output data set from the at least one optically detectable signal. In some embodiments, the aqueous solution comprises salts, polymer surfactants, buffers, and combinations thereof. In some embodiments, the step of enriching comprises heating the enrichment solution mixed with the eluate. In some embodiments, the detector comprises chromatography device. In some embodiments, the chromatography device is a lateral flow assay. In some embodiments, the sample has a volume comprising at most or about 400 microliters (μl), 350 μl, 300 μl. 250 μl, 200 μl, 150 μl, 100 μl, 50 μl, 45 μl, 40 μl, 35 μl, 30 μl or less. In some embodiments, the sample comprises an amount of the target analytes comprising between or about 4pg to 100pg, 4pg to 150pg, 4pg to 200pg, 4pg to 250pg, 4pg to 300pg, 4pg to 350pg, 4pg to 400pg, 4pg to 450pg, 4pg to 500pg, 10pg to 100pg, 10pg to 150pg, 10pg to 200pg, 10pg to 250pg, 10pg to 300pg, 10pg to 350pg, 10pg to 400pg, 10pg to 450pg, 10pg to 500pg, 20pg to 100pg, 20pg to 150pg, 20pg to 200pg, 20pg to 250pg, 20pg to 300pg, 20pg to 350pg, 20pg to 400pg, 20pg to 450pg, 20pg to 500pg, 30pg to 100pg, 30pg to 150pg, 30pg to 200pg, 30pg to 250pg, 30pg to 300pg, 30pg to 350pg, 30pg to 400pg, 30pg to 450pg, or 30pg to 500pg. In some embodiments, the cfDNA is fragmented.

Aspects disclosed herein provide methods for detection of cell-free DNA (cfDNA) in blood comprising: receiving a sample comprising whole blood; filtering the sample to substantially remove solid particles, the solid particles comprising red blood cells, white blood cells, apoptotic bodies, viral particles, or combinations thereof, thereby producing blood plasma; mixing the blood plasma with a first aqueous solution; binding cell-free DNA (cfDNA) molecules to a surface of one or more paramagnetic microspheres; separating the microspheres from the solution of the blood plasma and the first aqueous solution; washing the microspheres with a second aqueous solution; separating the microspheres from the cfDNA molecules using an elution, thereby producing an eluate comprising purified cfDNA molecules; enriching the eluate to increase a number of the cfDNA molecules; diluting the eluate with an aqueous buffer; introducing the eluate to a chromatographic paper strip thereby producing one or more optically detectable signals; and outputting a dataset comprising detection of the one or more optically detectable signals.

Aspects disclosed herein provide methods for detection of quantities of target antigens in blood comprising: receiving a sample comprising whole blood; filtering the sample to substantially remove solid particles, the solid particles comprising red blood cells, white blood cells, apoptotic bodies, or, viral particles, or combinations thereof, thereby producing blood plasma; mixing the blood plasma with a first aqueous solution; binding the target antigens to DNA labeled antibodies; binding the target antigens to primary antibodies coated on more microspheres, wherein the primary antibodies selectively bind to the target antigens thereby producing bound triads comprising a target antigens, a primary antibodies, and a DNA labeled antibodies; washing the bound triads and the microspheres with a second aqueous solution; removing the bound triads from the microspheres using an elution; enriching a solution containing the bound triads to produce an enriched solution; diluting the enriched solution with an aqueous buffer; introducing the enriched solution to a chromatographic paper strip thereby producing one or more optically detectable signals; and outputting a dataset comprising detection of the one or more optically detectable signals, wherein a quantity of the one or more optically detectable signals is proportional to a quantity of the target antigens in the enriched solution. In some embodiments, the sample has a volume comprising at most or about 400 microliters (μ1), 350 μl, 300 μl. 250 μl, 200 μl, 150 μl, 100 μl, 50 μl, 45 μl, 40 μl, 35 μl, 30 μl or less. In an embodiment, the sample comprises an amount of the cfDNA comprising between or about 4pg to 100pg, 4pg to 150pg, 4pg to 200pg, 4pg to 250pg, 4pg to 300pg, 4pg to 350pg, 4pg to 400pg, 4pg to 450pg, 4pg to 500pg, 10pg to 100pg, 10pg to 150pg, 10pg to 200pg, 10pg to 250pg, 10pg to 300pg, 10pg to 350pg, 10pg to 400pg, 10pg to 450pg, 10pg to 500pg, 20pg to 100pg, 20pg to 150pg, 20pg to 200pg, 20pg to 250pg, 20pg to 300pg, 20pg to 350pg, 20pg to 400pg, 20pg to 450pg, 20pg to 500pg, 30pg to 100pg, 30pg to 150pg, 30pg to 200pg, 30pg to 250pg, 30pg to 300pg, 30pg to 350pg, 30pg to 400pg, 30pg to 450pg, or 30pg to 500pg. In an embodiment, the cfDNA is fragmented.

Aspects disclosed herein provide methods for analyzing a quantity of target antigens in a sample comprising: removing solid particles from a sample using a filter, thereby producing a filtered sample; mixing the filtered sample with a first aqueous solution; contacting DNA labeled antibodies and primary antibodies attached to microspheres to the sample, wherein the sample comprises target antigens; binding the target antigens in the sample to the DNA labeled antibodies and the primary antibodies, thereby producing a conjugate solution comprising bound triads of a target antigen, a primary antibody, and a DNA labeled antibody, wherein the bound triad is attached to a microsphere; washing the conjugate solution with a second aqueous solution; removing the DNA labeled antibodies from the bound triads using an elution; enriching the one or more DNA labeled antibodies, thereby producing an enriched solution; diluting the enriched solution with an aqueous buffer; introducing the enriched solution to a chromatographic paper strip, thereby producing detectable signals; and outputting a dataset comprising detection of the detectable signals, wherein a quantity of the detectable signals is proportional to the quantity of the target antigens. In some embodiments, the sample is whole blood. In some embodiments, the sample has a volume comprising at most or about 400 microliters (μl), 350 μl, 300 μ1. 250 μl, 200 μl, 150 μl, 100 μl, 50 μl, 45 μl, 40 μl, 35 μl, 30 μl or less. In some embodiments, removing the solid particles comprises removing red blood cells, white blood cells, apoptotic bodies, or viral particles, or combinations thereof, from the sample, thereby producing blood plasma.

Aspects disclosed herein provide devices for preparing a sample for analyte detection comprising: a processor comprising a filter comprising a filter inlet to receive the sample and a filter outlet to output a filtered sample: and an enricher configured to increase a number of target analytes for detection; a fluid routing network comprising: a first fluid pathway coupling the filter outlet to the enricher; a first valve along the first fluid pathway; a second valve along the first fluid pathway; and a first fluid junction positioned between the first and second valves and coupling a first pump channel to the first fluid pathway; a fluid supply comprising: a first pump in fluid communication with the first pump channel and a first reservoir containing an aqueous solution, wherein the first pump is configured to supply the aqueous solution to the enricher and transport the filtered sample through the first fluid pathway; and an electronics and software subsystem that controls the first pump, the first valve, and the second valve. In some embodiments, the aqueous solution and the filtered sample are mixed in the enricher to form a sample solution, and wherein the sample solution is heated within the enricher to produce an enriched sample. In some embodiments, the enriched sample is output from the device by the first pump through an enricher outlet. In some embodiments, the aqueous solution comprises salts, polymer surfactants, buffers, or a combination thereof. In some embodiments, the processor further comprises a detector comprising a chromatography device. In some embodiments, the fluid routing network further comprises: a second fluid pathway coupling an outlet of the enricher to an inlet of the detector; a third valve along the second fluid pathway; a fourth valve along the second fluid pathway; and a second fluid junction in fluid communication with the second fluid pathway and positioned between the third and fourth valves. In some embodiments, the fluid supply further comprises: a second pump in fluid communication with the second fluid junction and a second reservoir containing an aqueous buffer, wherein the second pump is configured to supply the aqueous buffer to the second fluid junction; wherein: the electronics and software subsystem further controls the third valve, the fourth valve, and the second pump, the enriched sample and the aqueous buffer are mixed within the second fluid junction to produce a buffered sample, and the second pump transports the buffered sample to the inlet of the detector.

Aspects disclosed herein provide devices for preparing a sample for analyte detection comprising: a processor comprising: a filter comprising a filter inlet to receive the sample and a filter outlet to output a filtered sample; a hybridizer configured to receive the filtered sample and hybridize the filtered sample to produce a hybridized sample; an eluter configured to receive the hybridized sample and elute the hybridized sample to produce an eluate; and an enricher configured to increase a number of analytes in the eluate for detection, thereby producing an enriched sample; a fluid routing network comprising: a first fluid pathway coupling the filter outlet to the enricher; a first valve along the first fluid pathway; a second valve along the first fluid pathway; a first fluid junction positioned between the first and second valves and coupling a first pump channel to the first fluid pathway; a third valve along the first fluid pathway; a second fluid junction provided between the second and third valves, the second fluid junction coupling the first fluid pathway, a second pump channel, a third pump channel, and the hybridizer; a fourth valve along the first fluid pathway; a third fluid junction in fluid provided between the third and fourth valves and coupling the first fluid pathway with a fourth pump channel; a fifth valve along the first fluid pathway; a fourth fluid junction provided between the fourth and fifth valves and coupling the eluter to the first fluid pathway; a sixth valve along the first fluid pathway; a fifth fluid junction in provided between the fifth and sixth valves and coupling a fifth pump channel to the first fluid pathway; and a sixth fluid junction coupling the enricher and the first fluid pathway, wherein the fluid supply comprises: a first pump in fluid communication the first pump channel and a first reservoir containing an aqueous solution, wherein the first pump is configured to supply the aqueous solution to the hybridizer and transport the filtered sample through the first fluid pathway; a second pump in fluid communication with the second pump channel and a second reservoir containing a washing solution, wherein the second pump is supplies the washing solution to the hybridizer; a third pump in fluid communication with the third pump channel and a third reservoir, wherein the third pump is configured to remove the washing solution from the hybridizer and deposit the washing solution into the third reservoir; a fourth pump in fluid communication with the fourth pump channel and a fourth reservoir containing a solvent, wherein the fourth pump is configured to supply the solvent to the eluter and transport the hybridized sample through the first fluid pathway; and a fifth pump in fluid communication with the fifth pump channel and a fifth reservoir containing an enrichment solution, wherein the fifth pump is configured to supply the enrichment solution to the enricher and transport the eluate through the first fluid pathway. In some embodiments, the processor further comprises a diluter to dilute the enriched sample and produces a diluted sample; and wherein the fluid routing network further comprises: a second fluid pathway coupling the enricher to the diluter; a seventh valve along the second fluid pathway; an eighth valve along the second fluid pathway; a seventh fluid junction in provided between the seventh and eighth valves and coupling a sixth pump channel to the second fluid pathway; and an eighth fluid junction coupling the diluter to the second fluid pathway; and wherein the fluid supply further comprises a sixth pump in fluid communication with the sixth pump channel and a sixth reservoir containing an aqueous buffer, wherein the sixth pump is configured to supply the aqueous buffer to the diluter and transport the enriched sample through the second fluid pathway. In some embodiments, the processor further comprises a detector; wherein the fluid routing network further comprises: a third fluid pathway coupling the diluter to the detector; a ninth valve along the third fluid pathway; a tenth valve along the third fluid pathway; and a ninth fluid junction in provided between the ninth valves and tenth valves and coupling a seventh pump channel to the third fluid pathway; and wherein the pump supply further comprises a seventh pump in fluid communication the seventh pump channel, wherein the seventh pump is configured to transport the diluted sample through the third fluid pathway.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts component groups of a system for preparing samples for molecular analyte detection, according to a non-limiting embodiment.

FIG. 2 depicts an example network configuration of a system for preparing samples for molecular analyte detection, according to a non-limiting embodiment.

FIG. 3 depicts an example network configuration of a system for preparing samples for molecular analyte detection, according to a non-limiting embodiment.

FIG. 4 depicts an example network configuration of a system for preparing samples for molecular analyte detection, according to a non-limiting embodiment.

FIG. 5 depicts an example network configuration of a system for preparing samples for molecular analyte detection, according to a non-limiting embodiment.

FIG. 6 depicts an example network configuration of a system for preparing samples for molecular analyte detection, according to a non-limiting embodiment.

FIG. 7 depicts an example network configuration of a system for preparing samples for molecular analyte detection, according to a non-limiting embodiment.

FIG. 8 shows a schematic of a device configuration of a system for preparing samples for molecular analyte detection, according to a non-limiting embodiment.

FIG. 9 shows a schematic of a device of a system for preparing samples for molecular analyte detection, according to a non-limiting embodiment.

FIG. 10 shows a schematic of a device configuration of a system for preparing samples for molecular analyte detection, according to a non-limiting embodiment.

FIG. 11 shows a schematic of a module within a fluid storage an actuation subsystem of a system for preparing samples for molecular analyte detection, according to a non-limiting embodiment.

FIG. 12 shows a schematic of a module within a fluid storage an actuation subsystem of a system for preparing samples for molecular analyte detection, according to a non-limiting embodiment.

FIG. 13 shows a schematic of a module within a fluid storage an actuation subsystem of a system for preparing samples for molecular analyte detection, according to a non-limiting embodiment.

FIG. 14 depicts a topology of a fluid routing network of a system for preparing samples for molecular analyte detection, according to a non-limiting embodiment.

FIG. 15A depicts a topology of a fluid routing network of a system for preparing samples for molecular analyte detection, according to a non-limiting embodiment.

FIG. 15B depicts a topology of a fluid routing network of a system for preparing samples for molecular analyte detection, according to a non-limiting embodiment.

FIG. 16 depicts a process flow for a system for preparing samples for molecular analyte detection, according to a non-limiting embodiment.

FIG. 17 depicts a process flow for a system for preparing samples for molecular analyte detection, according to a non-limiting embodiment.

FIG. 18 depicts a process flow for a system for preparing samples for molecular analyte detection, according to a non-limiting embodiment.

FIG. 19 shows a schematic of a device configuration of a system for preparing samples for molecular analyte detection, according to a non-limiting embodiment.

FIG. 20 shows a schematic of a device configuration of a system for preparing samples for molecular analyte detection, according to a non-limiting embodiment.

FIG. 21 shows a time-variant normalized intensity function of lines developed across a later flow assay device strip, according to a non-limiting embodiment.

FIG. 22 depicts a cutoff value for a test signal, according to a non-limiting embodiment.

### DETAILED DESCRIPTION

Provided herein are systems and devices for preparing a sample for analyte detection. In an embodiment, a sampling device receives a sample and prepares the sample for analyte detection. In some embodiments, the sampling device includes a detection component to detect analytes of interest and produces a quantitative or qualitative data output. In some cases, the sampling device is a point of need (PON) device. In some instances, one or more sampling devices and systems disclosed herein communicate information about analytes of interest in a sample to a communication device or communication interface connected to a communication network.

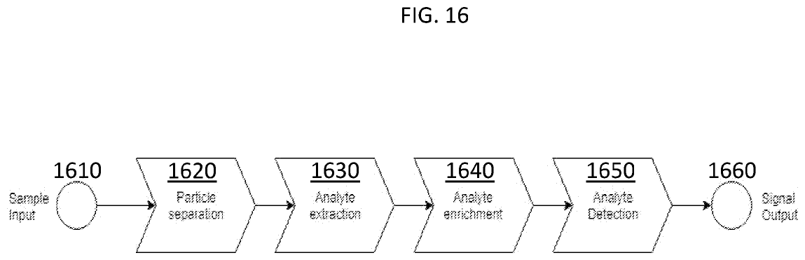

In an embodiment, the system automates a sequence of at least one materials processes that are traditionally executed manually or robotically in a laboratory environment. In this manner, a PON sampling device fully processes a sample to result without the explicit handling of liquids by users. The systems and devices of the present disclosure are particularly useful for the detection of DNA, RNA, or proteins in a sample, as exemplified in FIG. 16.

Existing sample processing and analyte detection methodologies suffer distinct disadvantages that limit broad applicability at PON. Table 1 provides a summary of the disadvantages of existing sample processing and analyte detection methodologies, in comparison with the devices and systems disclosed herein.

TABLE 1

| | Traditional | Robotic | Microfluidic | Present |
|---|---|---|---|---|
| | Processing | Processing | Device | Embodiments |
| Sample-to- | [symbol] | [symbol] | [symbol] | [symbol] |
| answer | | | | |
| Portable | [symbol] | [symbol] | [symbol] | [symbol] |
| Automated | [symbol] | [symbol] | [symbol] | [symbol] |
| Disposable | [symbol] | [symbol] | [symbol] | [symbol] |
| single unit | | | | |
| Non-technical/ | [symbol] | [symbol] | [symbol] | [symbol] |
| professional | | | | |
| operator | | | | |
| One system, | [symbol] | [symbol] | [symbol] | [symbol] |
| multiple | | | | |
| products | | | | |

Manual laboratory processing is the gold standard method of executing most diagnostic, clinical and research assays. Sample processing involves manually manipulating samples and liquid reagents with pipettes, disposable reaction media, processing machines (e.g. centrifuges, filtration systems), and detection instruments (e.g. mass spectrometers, plate readers). A human operator develops and executes protocols in a specialized laboratory environment optimized to enable their workflow, mitigate failures due to contamination and procedural errors, and mitigate safety risks. The manual nature by which assays are executed is time-consuming and error prone (human errors such as sample mix-ups, breakages, reagent mix-ups), with a typical nucleic acid amplification technology (NAT) based test (e.g. qPCR) taking up to 2 days.

Robotic laboratory processing is the use of robotic machines to automate protocols, that are traditionally executed manually. Although robotic laboratory processing reduces human error and data variation, and enables higher throughput processing, robots a cost prohibitive in most contests. Administrative, infrastructural, and engineering costs limits applicability only to central laboratory facilities responsible for processing a high volume of assays.

Microfluidic processing involves the manipulation of liquids at the nano-, micro- and millimeter scale. Microfluidic devices are limited by the technical difficulty and high cost in producing devices with more than one layer of channels for fluid flow. For this reason, there does not exist an integrated microfluidic device capable of sample processing (e.g., purification, amplification) and analyte detection. At most, microfluidic devices automate only part of the materials process flow for an assay and must interface with external instruments for detection and fluid actuation, thereby obviating their claimed benefits over manual laboratory processing.

Disclosed herein, in some embodiments, are devices and systems suitable PON application that are capable of sample processing (purification, amplification) and analyte detection. Turning to FIG. 16, in some embodiments, a sample is received by the sample input 1610. Particles are then extracted from the liquid input as a waste byproduct of the process at 1620. These particles are removed as they may interfere in future processing of the liquid component of the input sample or detection of molecular analytes of interest. In an embodiment, particle separation is carried out by a filter. In an embodiment, the filter is a polymer-based filter such as a nitrocellulose, plastic, or paper membrane. In another embodiment, the filter comprises a paper membrane. In an embodiment a paper membrane filter is sourced from Pall (“Vivid”) specifically manufactured for blood separation. Alternatively, the filter could be a woven mesh, weir (or dam) type structure, frit, array of microfabricated holes, porous gel, or packed bed of particles.

Next, analytes are extracted from the resultant solution at step 1630, generating a new solution in which they are present in a fully or partially purified form. The concentration of extracted analytes may be extremely low and therefore difficult or impossible to detect directly. The extracted analytes are then enriched to bring their quantity or concentration into detectable range at step 1640. Alternatively, the extracted analytes are introduced to reactants that act to amplify their detectable signal, such as an enzyme, dyed particle, or fluorophore. Finally, the enriched product is detected at step 1650 and one or more associated signals are produced as an output 1660.

### Sampling Device

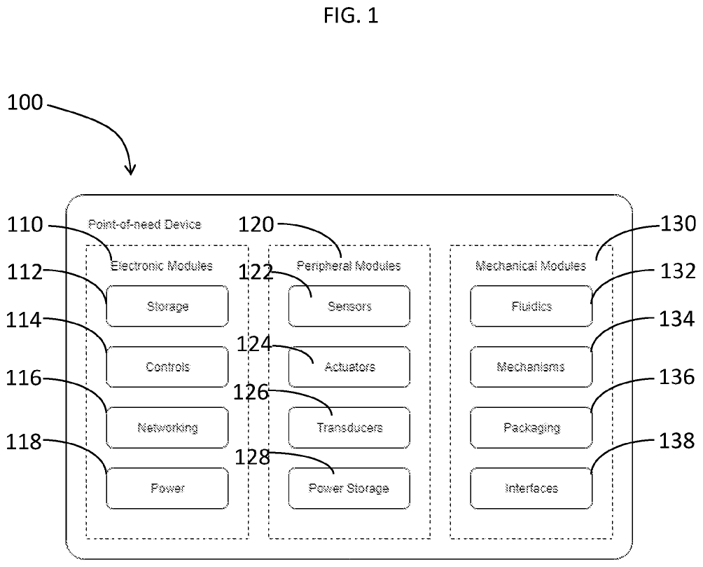

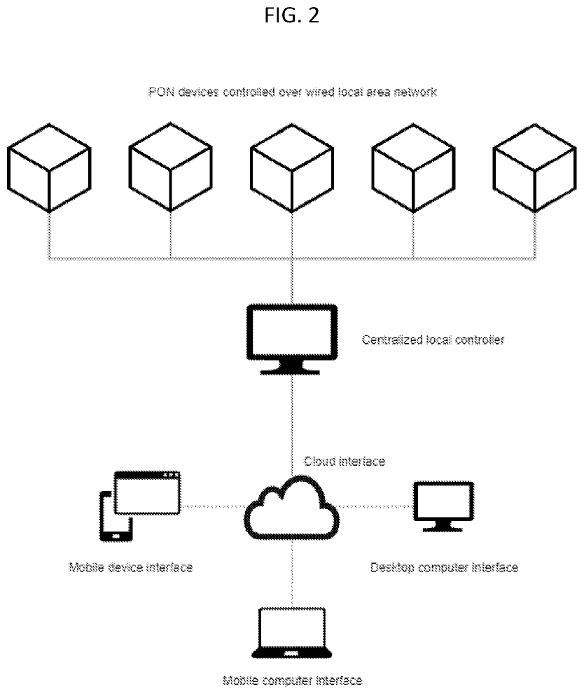

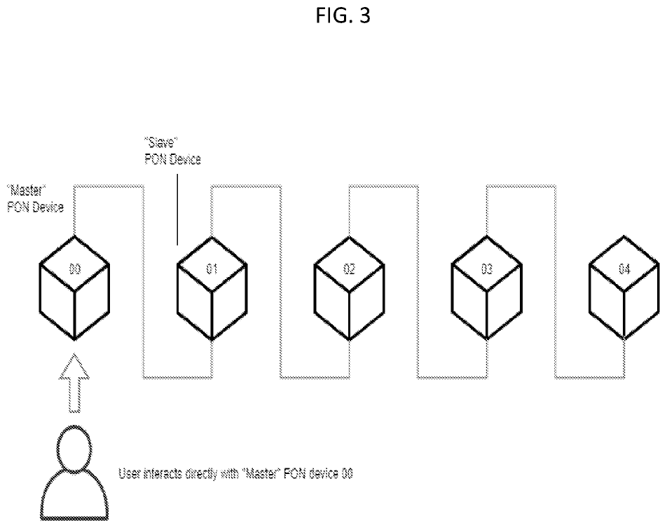

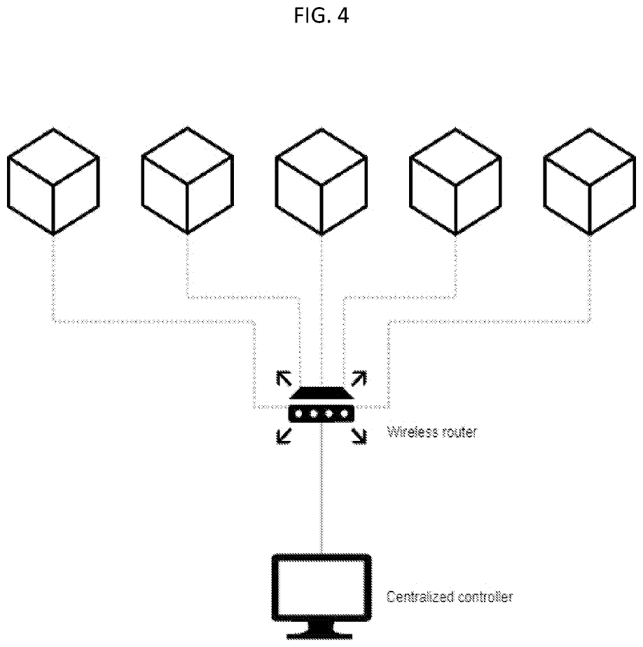

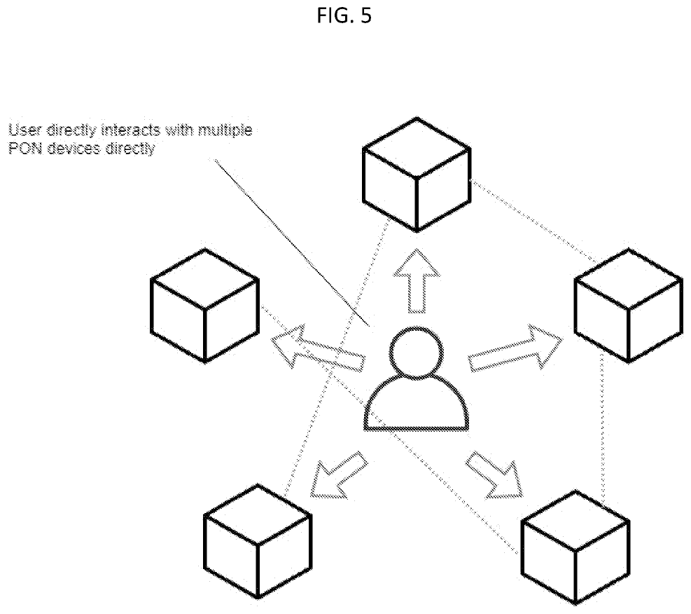

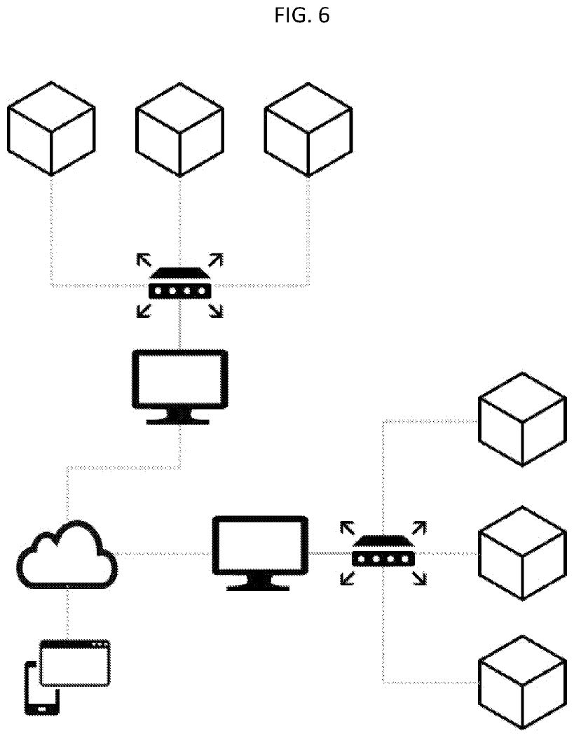

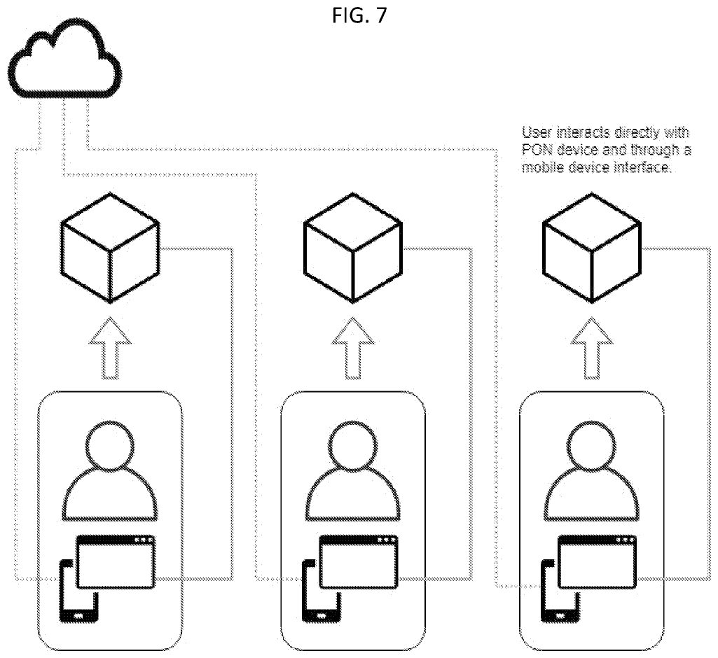

FIG. 1 is a visual representation of an embodiment of a point of need sampling device 100 and components thereof. These component groups are interconnected within a unit to automatically execute a sequence of materials processes. Hence, the electronic components within the unit generate, manage, and store data used to control or as a result from the motion of mechanical and peripheral components. The coordinated behavior of component groups may therefore be controlled entirely within one unit of the invention, though data may be transmitted and received over a network (depicted by FIG. 7). Transmitting data over the network is particularly useful in an environment where there is little or no laboratory infrastructure, such as in the home of a consumer or in medical emergency departments. Alternatively, an encompassing control system may be used to coordinate the behavior of electronic and mechanical components associated with multiple invention units. In such an embodiment, the encompassing control system generates, communicates, manages, and stores data from one or more sampling units 100 in a wired or wireless network. Such a configuration is particularly useful when the device is being used in a centralized laboratory or production environment to supplement materials processing executed through traditional manual and automated techniques. Embodiments of such a network include control of individual sampling units over a local area network (FIG. 2), master-slave serial connection between sampling units (FIG. 3), wireless local area networks (FIG. 4), ad hoc networks (FIG. 5), and cloud-based networks (FIG. 6).

In an embodiment, the point of need sampling device comprises electronic modules 110. In an embodiment, the electronic modules 110 include a storage module for storing data such as detected signals, processing parameters, and fluids levels. In an embodiment, the electronic modules further comprise a controls module 114, for controlling timing and activation of electronic components of the system such as valves, pumps, electromagnets, and heaters. In an embodiment, the electronic modules 110 comprise a networking module 116 to connect to other sampling devices, a controller, a remote interface, or a combination thereof. In an embodiment, the electronic modules 110 of the sampling device 100 comprise a power module 118. The power module 118 regulates and supplies power to electronic components of the system.

In an embodiment, the point of need sampling device 100 comprises one or more peripheral modules 120. In an embodiment, the peripheral modules include actuator 124, transducer 126, and sensor 122 modules to monitor and record data during sample processing. Exemplary sensors include temperature sensors, pH sensors, fluid level sensors, pressure sensors, and other sensors suitable for use in the sampling device 100. In an embodiment, the peripheral modules 120 may further include one or more power storage modules 128. Power storage modules 128 may include primary type (single-use) batteries, secondary type (rechargeable) batteries, fuel cells, and supercapacitors to power the sampling device 100 or components thereof.

In an embodiment, the point of need device comprises mechanical modules 130 which include fluidics systems 130. Fluid systems 130 are provided to transport a sample through the device 100 and supply reagents to modules of the sampling device 100. Mechanisms 134 such as valves and pumps are actuated in the sampling device 100 to control transportation of a sample through the device, mixing of reagents, supply of reagents to modules of the device, and removal of fluid and by product from modules of the device.

In an embodiment, the sampling device includes an enclosure or packaging 136 to contain all the elements of the sampling device. In some embodiments, the packaging further includes labels throughout, on its surface, or stored in its memory. The labels may be encoded with data relevant to a unit of the invention's manufacture, operation, distribution, sale, disposal, or recycling. Example labels include barcodes, QR codes, and other printed labels that can be recognized optically. Other example labels include color change stickers such as those indicating temperature, humidity, pressure, vibration, radiation, and other physical strain a unit of the invention may be exposed to. Digital labels can include RFID devices and other network modules with local memory or stored in the main controller memory; digital labels include data not directly relevant to the execution of the assay itself but are transmittable over a network. In some embodiments, components and subassemblies are labeled internally for manufacturing controls and supply chain management purposes. This includes manufacturer-applied labels as well as device-unique labels. These labels may identify the components or some attribute of the components, such as lot number, date of production, or other keys or links relevant to database systems. Furthermore, labels such as when, where, and by whom the test was first activated can be generated in real time and transmitted over a network. These data could be accessed for purposes of managing clinical trials, product design, and user studies. Combining operation and manufacturing labels could enable logistical tracking for distribution purposes as well, such as managing shipping for units with a limited shelf-life.

In an embodiment, a sampling devices comprises one or more mechanical or electrical interfaces 138 which couple the three distinct subsystems of each sampling device.

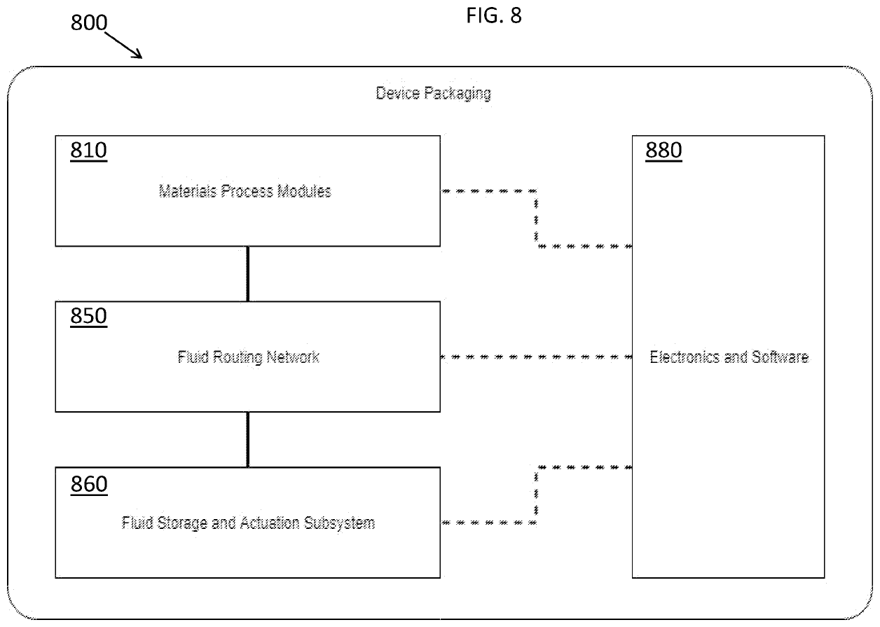

In an embodiment, a sampling device 800 comprises three distinct subsystems that interface with one another mechanically and electronically (FIG. 8): a system of materials process modules (“MPM”) 810, a fluidic routing network (“FRN”) 850, a fluid storage and actuation system (“FSA”) 860, and electronics and software 880. The processor (MPM) 810 and fluid supply (FSA) 860 are connected to the routing network (FRN) 850 through one or more fluidic pathways and mechanical couplings. The electronics and software 880 are connected to the MPM 810, FSA 860, and FRN 850. The purpose of the FRN 850 is to direct fluids to and from modules in the FSA 860 and MPM 810. The purpose of the MPM 810 is to execute critical materials processing steps that are traditionally executed using instruments and disposables in laboratory environments. The purpose of the FSA 860 is to manage reagents critical to the automated materials process flow. The purpose of the electronics and software 880 is to coordinate active devices in the MPM 810, FSA 860, and FRN 850 such that the invention can execute a materials process flows in a fully or partially automated manner.

In some aspects, disclosed herein are sampling devices and systems for obtaining genetic information from a biological sample. As described herein, sampling devices and systems disclosed herein allow a user to collect and test a biological sample at a location of choice to detect the presence and/or quantity of a target analyte in the sample. In some instances, sampling devices and systems disclosed herein are used in the foregoing methods. In some instances, sampling devices and systems disclosed herein comprise a sample purifier, filter, eluter, or a combination thereof that removes at least one component (e.g., cell, cell fragment, protein) from a biological sample of a subject; a hybridizer or nucleic acid sequencer for sequencing at least one nucleic acid in the biological sample; and a detection device or nucleic acid sequence output for relaying sequence information to a user of the device, system or kit.

In an embedment, the materials processor or materials process modules subsystem (MPM) is composed of one or more modules that manage fluids and chemical reactions. Each module in the MPM may add or remove energy from fluids within it. Examples forms of energy include thermal, acoustic, mechanical, chemical, particle radiation, and electromagnetic radiation. The energy may be generated by an instrument external to the invention or a device integrated within the assembly. Examples of the latter include thermoelectric coolers, heating filaments, laser diodes, electrodes, antennae, ultrasonic speakers, photovoltaic diodes, radioactive masses, and phototransistors. In this manner, a module in the MPM may interface fluids with transducers, sensors, and actuators. A module in the MPM may also therefore receive an input fluid sample or create an output detectable signal. Each module in the MPM may add or remove fluid components or engage in transporting fluids. Example functions of adding or removing fluid components include filtration, precipitation, chromatography, affinity-based separation, mixing, volumetric metering, and phase separation. Example methods of transporting fluids include pumping and storage in a reservoir. Modules in the MPM commonly have one or more inlets through which fluids involved in the automated materials process flow may enter or exit. They also commonly have one or more inlets through which a fluid not involved with the automated materials process flow may enter or exit. For example, as liquids enter a module in the MPM, air may be displaced proportional to its volume through a vent. In another example, an immiscible working liquid may be used to pressurize fluid that is stored within the module, initiating a chemical reaction.

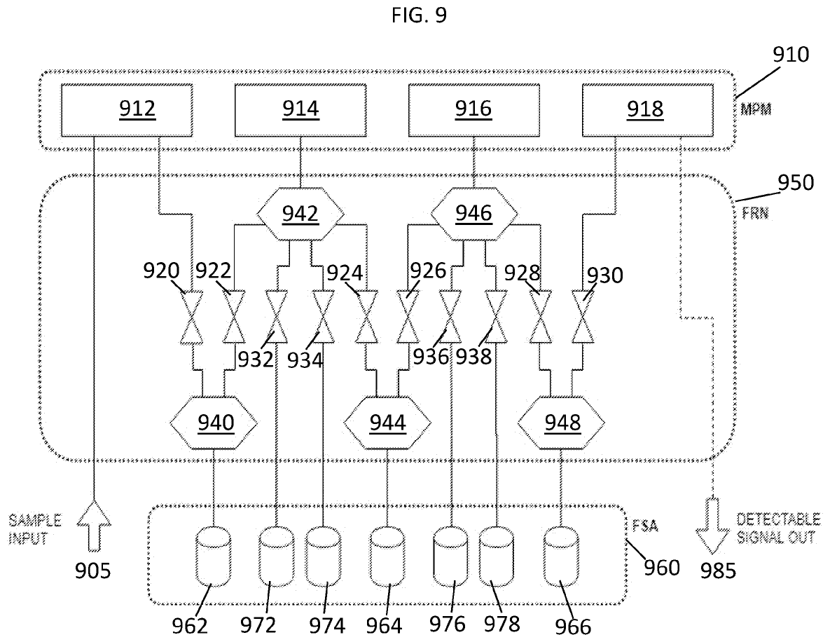

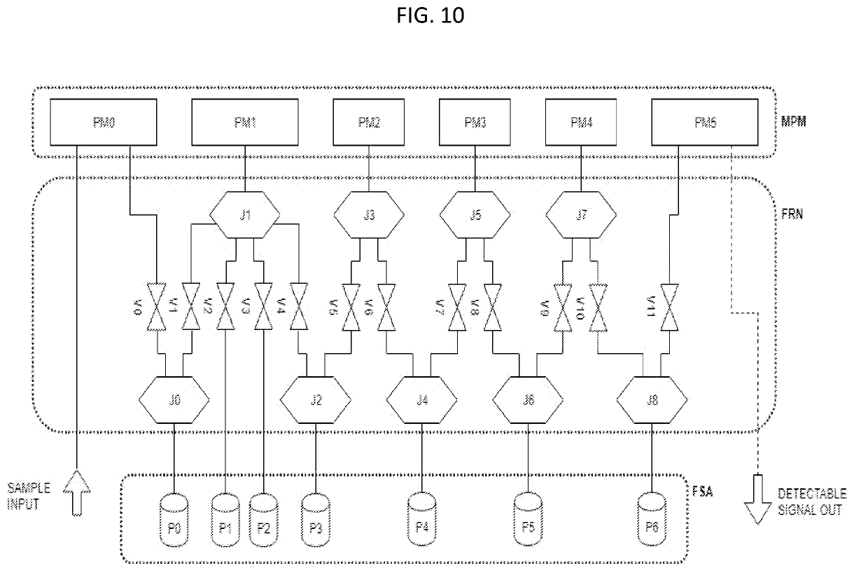

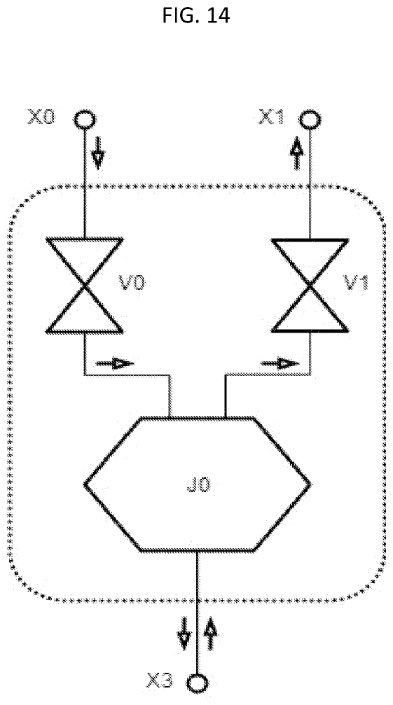

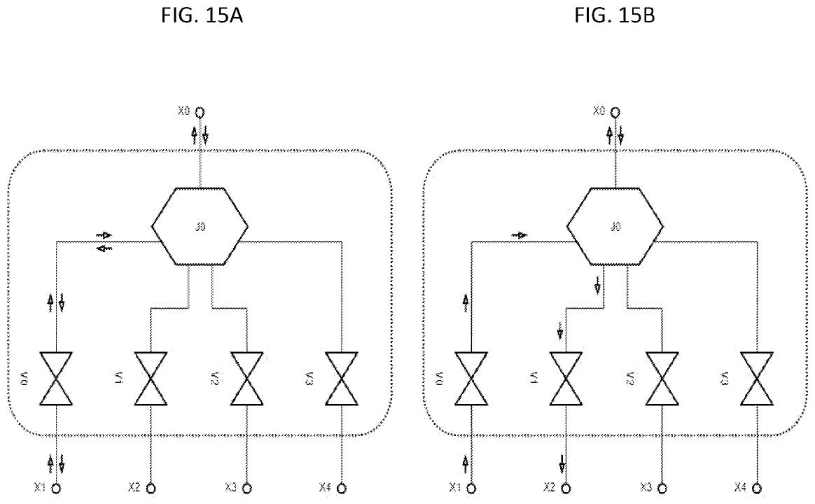

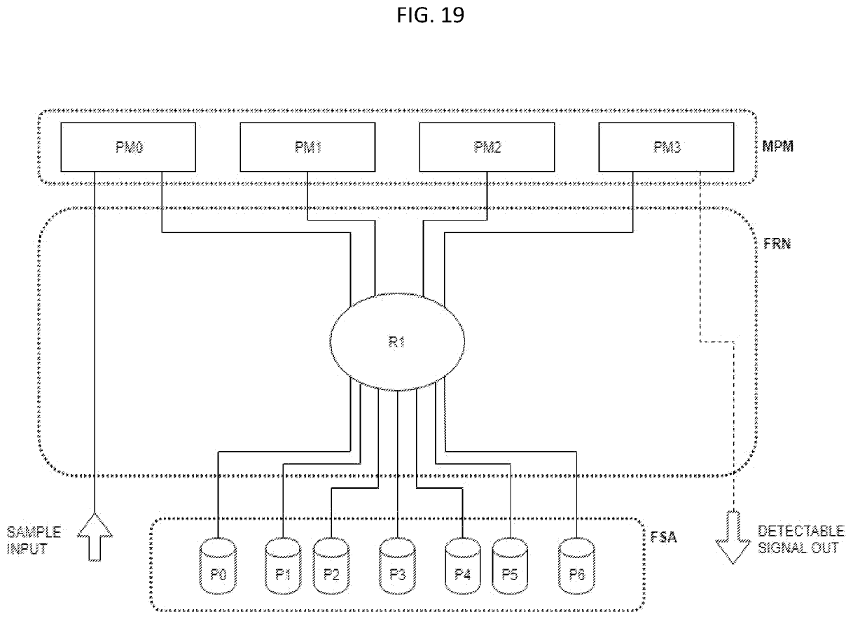

In an embodiment, the fluidics routing network (FRN) is composed of one or more fluidic pathways and valves. Example structures containing fluidic pathways include tubing, diffusion bonded manifolds, manifolds containing drilled channels, and microfluidic devices. Fluidic pathways can be devices with only two inlets such as a straight tube or more than two inlets such as wye junctions. Example valves include pinch valves, diaphragm valves, isolation valves, rotary valves, check valves, and tesla valves. Three examples of FRN fluid flow network topologies are given in FIG. 9, FIG. 10, and FIG. 19. In all three examples, the sample is added into the invention and an output signal is created via modules in the materials processing module (MPM). In the first two examples, two common topologies are apparent within the FRN itself. The first common topology enables unidirectional flow from one node to another typically in the MPM through a single node containing bidirectional flow typically in the FSA (FIG. 14). In this manner, fluid can be moved from a module in the MPM to a module in the FSA where it is potentially combined with new fluids, mixed, or stored in part. Then it can be moved to a different module in the MPM for further processing. The second common topology enables uni- or bidirectional flow in nodes typically in the FSA to a single node containing bidirectional flow typically in the MPM FIG. 15. In this manner, fluids can be moved from a module in the FSA to a module in the MPM where they are processed. The resultant fluid can then be returned to the originating module in the FSA where it is stored or treated as waste. Alternatively, the resultant fluid can be returned to a new destination module in the FSA where it can be combined with new fluids, mixed, or stored. This topology can also be used to simply transfer fluids from one module to another in the FSA if the interstitial destination node in the MPM does not act on the fluid beyond behaving as a reservoir. In this manner, multiple fluids can be removed from storage, mixed, and incubated in the FSA.

In an embodiment, FIG. 14 shows a network diagram depicting a common topology used in the fluidics routing network (FRN). Fluidic connections are indicated by solid lines and flow paths indicated by small arrows. Fluid is transported unidirectionally in two nodes typically located in the MPM labeled as X0 and X1. Fluid is transported bidirectionally in a single node in the FSA labeled as X3. Flow is ultimately directed from X0 to X1 through valves V0 and V1 and junction J0.

In an embodiment, FIGS. 15A and 15B show network diagrams depicting a common topologies used in the fluidics routing network (FRN). Fluid is transported bidirectionally in a node near the MPM labeled as X0. Flow is directed bidirectionally or unidirectionally through junction J0 and valves V0, V1, V2, and V3 to destination nodes in the FSA labeled as X1, X2, X3, and X4. An example of bidirectional flow in a destination node in the FSA is depicted in FIG. 15A. An example of unidirectional flow in a destination node in the FSA is depicted in FIG. 15B.

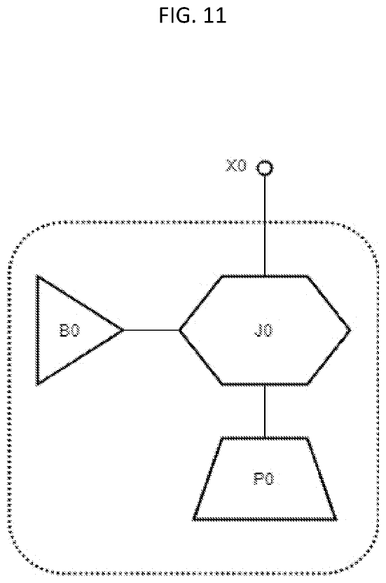

The fluid storage and actuation subsystem is comprised of one or more of fluid supply modules that store, mix, and/or pump fluids. In an embodiment, each fluid supply module comprises one or more devices that enable these functions. With reference to FIGS. 11-13 example topographies of fluid supply modules within the fluid storage and actuation subsystem are depicted. Example devices for pumping contained by modules in the FSA include syringe pumps, peristaltic pumps, fixed displacement pumps, and turbines. In addition, pumps may be realized through the combination of certain valves, including check valves, tesla valves, pinch valves, and diaphragm valves. Example devices for fluid storage include ultrasonic, pressure, adhesive, or thermally sealed containers, bladders, tubing, microfluidic reservoirs, syringes, and blisters. Example devices for mixing fluids include stir bars, acoustic transducers, vibrational motors, syringe pumps, microfluidic grooved channel structures, microfluidic herringbone channel structures, microfluidic helical channel structures, microfluidic lamination channel structures, split-and-recombine structures, and surface energy gradient structures. Furthermore, a fluid containing particles within a module in the FSA may be mixed by exerting a force on those particles. For example, if the particles have a differing magnetic susceptibility from the surrounding fluid medium, a magnetophoretic force caused by an arrangement of electromagnets near the container walls may be used to circulate a flow.

According to an embodiment, FIG. 11 depicts an example arrangement of devices within one fluid supply module 1100 of the fluid supply and actuation subsystem. Fluidic connections are indicated by solid lines. A single pump 1110 manages fluid flow and mixing. A single-use storage container 1120 releases fluid into the network upon activation through a junction 1130 while pump 1110 is acting as a closed circuit or source of withdrawal and node 1140 is acting as an open circuit. Fluids enter and exit the module 1100 from a single node 1140 inside the fluidic routing network subsystem.

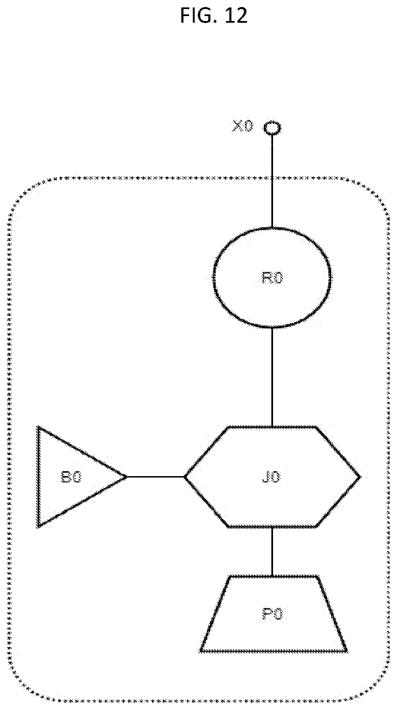

According to an embodiment, FIG. 12 depicts an example arrangement of devices within one fluid supply module of the fluid supply and actuation subsystem. Network diagram depicting an example arrangement of devices within one module of the FSA. Fluidic connections are indicated by solid lines. A single pump P0 manages fluid flow and mixing. A single-use storage container B0 releases fluid into the network upon activation through a junction J0. The fluids fill reservoir R0 while pump P0 is an open circuit and X0 is a closed circuit. Fluids enter and exit the module from a single node X0 inside the FRN.

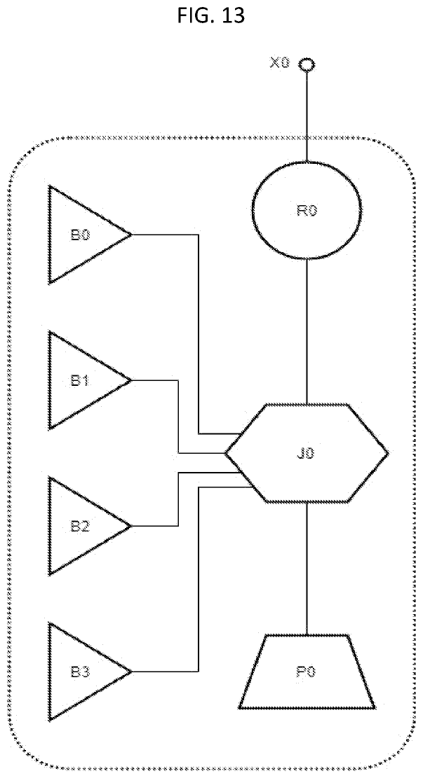

According to an embodiment, FIG. 13 depicts an example arrangement of devices within one fluid supply module of the fluid supply and actuation subsystem. Network diagram depicting an example arrangement of devices within one module of the FSA. Fluidic connections are indicated by solid lines. A single pump P0 manages fluid flow and mixing. Multiple single-use storage containers B0, B1, B2, and B3 release fluid into the network upon activation through a junction J0. The fluids fill reservoir R0 while pump P0 is an open circuit and X0 is a closed circuit. Fluids enter and exit the module from a single node XO inside the FRN.

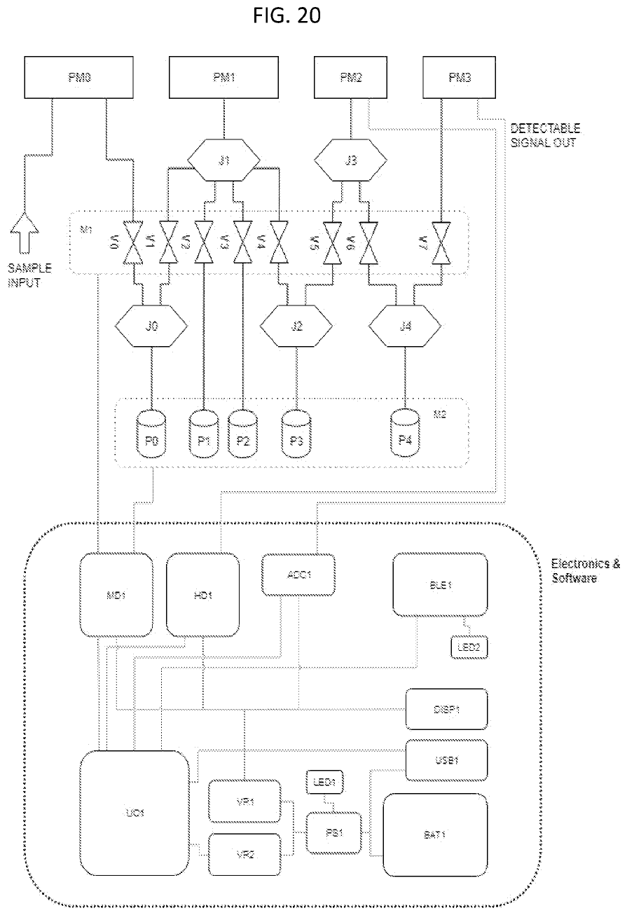

In an embodiment, the sampling device further comprises an electronics and software subsystem. Electronic devices and software of the subsystem serve to coordinate automated execution of the materials process flow and communicate data with the outside world. An example of one such system is presented in FIG. 20. Several classes of electronic devices may be present in the system, including those for power storage, power management, controls, memory, networking, sensing, signal manipulation, actuation, transduction, displaying information, and human interfacing. Power storage devices may include primary type (single-use) batteries, secondary type (rechargeable) batteries, fuel cells, and supercapacitors. Power management systems receive and regulate power from storages systems or from a wired connection to an external power source. Example external power sources include wall/grid power, mobile phones, generators, and solar cells. Networking devices allow for data such as those generated onboard sensors to be communicated to and from from external devices. Example networking devices include cell phone communication modules, Bluetooth modules, Zigbee modules, radio communications modules, near-field communication modules, and serial bus modules. Controls systems may be composed of one or more discrete elements or integrated circuits, including microcontrollers, embedded computers, programmable logic devices, field programmable gate array devices, analog feedback control devices, motor controllers, temperature controllers, and application specific integrated circuits. Devices for displaying information include electroluminescent displays, liquid crystal displays, light emitting diodes, light emitting diode displays, tickertape, rollfilm, and lamps. The invention may also contain other devices for human interaction, such as speakers, photodiode receivers, cameras, electrodes, microphones, buttons, touchpads, knobs, levers, and dials.

By way of non-limiting example, the user may be a pregnant subject and the region of interest may be a region on a Y chromosome. By way of non-limiting example, a region of interest may be in a gene implicated in a cancer, an autoimmune condition, a neurological disorder, a metabolic disorder, a cardiovascular disease, immunity (e.g., infection susceptibility or resistance), and drug metabolism. A gene implicated in a disease, disorder or condition is considered a gene that when mutated, deleted, copied, epigenetically modified, under- or overexpressed, changes at least one of a symptom, outcome, duration, or onset of the disease, disorder or condition.

In general, sampling devices and systems of the present disclosure, integrate multiple functions, e.g., purification or filter, amplification or enrichment, and detection of the target analyte (e.g., including amplification products thereof), and combinations thereof. In some embodiments, the multiple functions are carried out within a single assay assembly unit or a single device. In some embodiments, all of the functions occur outside of the single unit or device. In some embodiments, at least one of the functions occurs outside of the single unit or device. In some embodiments, only one of the functions occurs outside of the single unit or device. In some embodiments, the sample purifier, nucleic acid amplification reagent, oligonucleotide, and detection reagent or component are housed in a single device. In general, sampling devices and systems of the present disclosure comprise a display, a connection to a display, or a communication to a display for relaying information about the biological sample to one or more people.

In some embodiments, sampling devices and systems comprise an additional component disclosed herein. Non-limiting examples of an additional component include a sample transportation compartment, a sample storage compartment, a sample and/or reagent receptacle, a temperature indicator, an electronic port, a communication connection, a communication device, a sample collection device, and a housing unit. In some embodiments, the additional component is integrated with the device. In some embodiments, the additional component is not integrated with the device. In some embodiments, the additional component is housed with the sample purifier, nucleic acid amplification reagent, oligonucleotide, and detection reagent or component in a single device. In some embodiments, the additional component is not housed within the single device.

In some embodiments, sampling devices and systems disclosed herein comprise components to obtain a sample, extract cell-free nucleic acids, and purify cell-free nucleic acids. In some embodiments, sampling devices and systems disclosed herein comprise components to obtain a sample, extract cell-free nucleic acids, purify cell-free nucleic acids, and prepare a library of the cell-free nucleic acids. In some embodiments, sampling devices and systems disclosed herein comprise components to obtain a sample, extract cell-free nucleic acids, purify cell-free nucleic acids, and sequence cell-free nucleic acids. In some embodiments, sampling devices and systems disclosed herein comprise components to obtain a sample, extract cell-free nucleic acids, purify cell-free nucleic acids, prepare a library of the cell-free nucleic acids, and sequence the cell-free nucleic acids. By way of non-limiting example, components for obtaining a sample are a transdermal puncture device and a filter for obtaining plasma from blood. Also, by way of non-limiting example, components for extracting and purifying cell-free nucleic acids comprise buffers, beads and magnets. Buffers, beads and magnets can be supplied at volumes appropriate for receiving a general sample volume from a finger prick (e.g., 50-150 μl of blood).

In some embodiments, sampling devices and systems comprise a receptacle for receiving the biological sample. The receptacle can be configured to hold a volume of a biological sample between 1 μl and 1 ml. The receptacle can be configured to hold a volume of a biological sample between 1 μl and 500 μl. The receptacle can be configured to hold a volume of a biological sample between 1 μl and 200 μl. In some embodiments, the receptacle is configured to hold less than 500 μL of whole blood. In some embodiments, the receptacle is configured to hold less than 400 μL of whole blood. In some embodiments, the receptacle is configured to hold less than 300 μL of whole blood. In some embodiments, the receptacle is configured to hold less than 200 μL of whole blood. In some embodiments, the receptacle is configured to hold less than 150 μL of whole blood. In some embodiments, the receptacle is configured to hold less than 100 μL of whole blood. In some embodiments, the receptacle is configured to hold less than 50 μL of whole blood. In some embodiments, the receptacle is configured to hold less than 400 μL of whole blood. In some embodiments, the receptacle is configured to hold less than 30 μL of whole blood.

In some embodiments, the receptacle is configured to hold at most or about 500 μL of whole blood. In some embodiments, the receptacle is configured to hold at most or about 400 μL of whole blood. In some embodiments, the receptacle is configured to hold at most or about 300 μL of whole blood. In some embodiments, the receptacle is configured to hold at most or about 200 μL of whole blood. In some embodiments, the receptacle is configured to hold at most or about 150 μL of whole blood. In some embodiments, the receptacle is configured to hold at most or about 100 μL of whole blood. In some embodiments, the receptacle is configured to hold at most or about 50 μL of whole blood. In some embodiments, the receptacle is configured to hold at most or about 400 μL of whole blood. In some embodiments, the receptacle is configured to hold at most or about 30 μL of whole blood.

The receptacle can have a defined volume that is the same as a suitable volume of sample for processing and analysis by the rest of the device/system components. This would preclude the need for a user of the device, system or kit to measure out a specified volume of the sample. The user would only need to fill the receptacle and thereby be assured that the appropriate volume of sample had been delivered to the device/system. In some embodiments, sampling devices and systems do not comprise a receptacle for receiving the biological sample. In some embodiments, the sample purifier receives the biological sample directly. Similar to the description above for the receptacle, the sample purifier can have a defined volume that is suitable for processing and analysis by the rest of the device/system components. In general, sampling devices and systems disclosed herein are intended to be used entirely at point of care. However, in some embodiments, the user can want to preserve or send the analyzed sample to another location (e.g., lab, clinic) for additional analysis or confirmation of results obtained at point of care. By way of non-limiting example, the device/system can separate plasma from blood. The plasma can be analyzed at point of care and the cells from the blood shipped to another location for analysis. In some embodiments, sampling devices and systems comprise a transport compartment or storage compartment for these purposes. The transport compartment or storage compartment can be capable of containing a biological sample, a component thereof, or a portion thereof. The transport compartment or storage compartment can be capable of containing the biological sample, portion thereof, or component thereof, during transit to a site remote to the immediate user. The transport compartment or storage compartment can be capable of containing cells that are removed from a biological sample, so that the cells can be sent to a site remote to the immediate user for testing. Non-limiting examples of a site remote to the immediate user can be a laboratory or a clinic when the immediate user is at home. In some embodiments, the home does not have a machine or additional device to perform an additional analysis of the biological sample. The transport compartment or storage compartment can be capable of containing a product of a reaction or process that result from adding the biological sample to the device. In some embodiments, the product of the reaction or process is a nucleic acid amplification product or a reverse transcription product. In some embodiments, the product of the reaction or process is a biological sample component bound to a binding moiety described herein. The biological sample component can comprise a nucleic acid, a cell fragment, an extracellular vesicle, a protein, a peptide, a sterol, a lipid, a vitamin, or glucose, any of which can be analyzed at a remote location to the user. In some embodiments, the transport compartment or storage compartment comprises an absorption pad, a paper, a glass container, a plastic container, a polymer matrix, a liquid solution, a gel, a preservative, or a combination thereof. An absorption pad or a paper can be useful for stabilizing and transporting a dried biological fluid with a protein or other biomarker for screening.

In some embodiments, sampling devices and systems disclosed herein provide for analysis of cell-free nucleic acids (e.g., circulating RNA and/or DNA) and non-nucleic acid components of a sample. Analysis of both cell-free nucleic acids and non-nucleic acid components can both occur at a point of need. In some embodiments, systems and devices provide an analysis of cell-free nucleic acids at a point of need and preservation of at least a portion or component of the sample for analysis of non-nucleic acid components at a site remote from the point of need. In some embodiments, systems and devices provide an analysis of non-nucleic acid components at a point of need and preservation of at least a portion or component of the sample for analysis of cell-free nucleic acids at a site remote from the point of need. These sampling devices and systems may be useful for carrier testing and detecting inherited diseases, such as those disclosed herein.

In some embodiments, the transport compartment or storage compartment comprises a preservative. The preservative can also be referred to herein as a stabilizer or biological stabilizer. In some embodiments, the device, system or kit comprises a preservative that reduces enzymatic activity during storage and/or transportation. In some embodiments, the preservative is a whole blood preservative. Non-limiting examples of whole blood preservatives, or components thereof, are glucose, adenine, citric acid, trisodium citrate, dextrose, sodium di-phosphate, and monobasic sodium phosphate. In some embodiments, the preservative comprises EDTA. EDTA can reduce enzymatic activity that would otherwise degrade nucleic acids. In some embodiments, the preservative comprises formaldehyde. In some embodiments, the preservative is a known derivative of formaldehyde. Formaldehyde, or a derivative thereof, can cross link proteins and therefore stabilize cells and prevent cell lysis.

In general, sampling devices and systems disclosed herein are intended to be used entirely at point of care. However, in some embodiments, the user may want to preserve or send the analyzed sample to another location (e.g., lab, clinic) for additional analysis or confirmation of results obtained at point of care. In some embodiments, sampling devices and systems comprise a transport compartment or storage compartment for these purposes. The transport compartment or storage compartment may be capable of containing a biological sample, a component thereof, or a portion thereof. The transport compartment or storage compartment may be capable of containing the biological sample, portion thereof, or component thereof, during transit to a site remote to the immediate user. Non-limiting examples of a site remote to the immediate user may be a laboratory or a clinic when the immediate user is at home. In some embodiments, the home does not have a machine or additional device to perform an additional analysis of the biological sample. The transport compartment or storage compartment may be capable of containing a product of a reaction or process that occurs in the device. In some embodiments, the product of the reaction or process is a nucleic acid amplification product or a reverse transcription product. In some embodiments, the product of the reaction or process is a biological sample component bound to a binding moiety described herein. The biological sample component may comprise a nucleic acid, cell fragment, an extracellular vesicle, a protein, a peptide, a sterol, a lipid, a vitamin, or glucose, any of which may be analyzed at a remote location to the user. In some embodiments, the transport compartment or storage compartment comprises an absorption pad, a paper, a glass container, a plastic container, a polymer matrix, a liquid solution, a gel, a preservative, or a combination thereof. In some embodiments, the device, system or kit comprises a stabilizer (chemical or structure (e.g., matrix)) that reduces enzymatic activity during storage and/or transportation.

Generally, sampling devices and systems disclosed herein are portable for a single person. In some embodiments, sampling devices and systems are handheld. In some embodiments, sampling devices and systems have a maximum length, maximum width or maximum height. In some embodiments, sampling devices and systems are housed in a single unit having a maximum length, maximum width or maximum height. In some embodiments the maximum length is not greater than 12 inches. In some embodiments the maximum length is not greater than 10 inches. In some embodiments the maximum length is not greater than 8 inches. In some embodiments the maximum length is not greater than 6 inches. In some embodiments the maximum width is not greater than 12 inches. In some embodiments the maximum width is not greater than 10 inches. In some embodiments the maximum width is not greater than 8 inches. In some embodiments the maximum width is not greater than 6 inches. In some embodiments the maximum width is not greater than 4 inches. In some embodiments the maximum height is not greater than 12 inches. In some embodiments the maximum height is not greater than 10 inches. In some embodiments the maximum height is not greater than 8 inches. In some embodiments the maximum height is not greater than 6 inches. In some embodiments the maximum height is not greater than 4 inches. In some embodiments the maximum height is not greater than 2 inches. In some embodiments the maximum height is not greater than 1 inch.

In some embodiments, sampling devices and systems disclosed herein comprise (a) a sample purifier that removes a cell from a biological fluid sample of a user subject; (b) at least one nucleic acid amplification reagent; (c) at least one oligonucleotide comprising a sequence corresponding to a region of interest, wherein the at least one oligonucleotide and nucleic acid amplification reagent are capable of producing an amplification product; and (d) at least one of a detection reagent or a signal detector for detecting the amplification product. In some embodiments, sampling devices and systems disclosed herein comprise a miniaturized digital nucleic acid amplification platform. By way of non-limiting example, the miniaturized nucleic acid amplification platform may be located on a chip within a device disclose herein, thereby keeping the entire device or system to a handheld size (e.g., similar to a cell phone). In some embodiments, the miniaturized nucleic acid amplification platform incorporates or is accompanied by digital output for ease of test result display.

In some embodiments, sampling devices and systems disclosed herein comprise (a) a sample purifier that removes a cell from a biological sample of a subject; (b) a nucleic acid sequencer for obtaining sequencing reads from nucleic acids in the biological sample; and (c) at least one of a detection reagent or a signal detector for detecting the sequencing reads. Non-limiting examples of a nucleic acid sequencer include next generation sequencing machines, nanopore sequencers, single molecule counters (e.g., counting sequences that are bar-coded/tagged).

The selection of materials that contact the sample is of significant importance. Poor choice of contact materials in the MPM, FRN, and FSA can lead to a poor or undetectable signal output over the expected contact time determined by the automated materials process and device lifetime requirements. Contacted materials must not desorb contamination or foreign substances into the fluid that inhibit chemical reactions executed in the MPM or FSA necessary for producing a detectable signal output. Contacted materials must not reduce the quantity of analyte through adsorption or absorption so severely that a detectable signal output cannot be produced. Contacted materials must not react with fluids or their components that are critical to the production of a detectable signal output. Contacted materials must not break down or change form in the presence of fluids such that their mechanical integrity is compromised so severely that a detectable signal output cannot be produced. In some embodiments, the contacted materials are solid polymers, such as polycarbonate, polypropylene, polystyrene, polyether ether ketone (PEEK), polytetrafluoroethylene (PTFE), polydimethylsiloxane (PDMS), and polyethylene. In some embodiments, the contacted materials are blends of polymers and composites, including but not limited to photoresins, thermoplastics, epoxies, and multimaterial constructs produced with additive manufacturing. In some embodiments, contacted materials are metals and metal alloys, such as stainless steel, anodized aluminum, copper, and inconel. In some embodiments, contacted materials are semiconductors or ceramics, such as silicon, germanium, silica, silicon nitride, gallium arsenide, and quartz.

### Sample Collection

Disclosed herein, in some embodiments, are systems and devices (e.g., sampling devices) configured to collect a sample from a subject. In some embodiments, the sample is whole blood, such as capillary blood. In some embodiments, the sample collection is performed by a sample collector comprising at transdermal puncture device.

In some embodiments, the sample comprises at most or about 500 μL of whole blood. In some embodiments, the sample comprises at most or about 400 μL of whole blood. In some embodiments, the sample comprises at most or about 300 μL of whole blood. In some embodiments, the sample comprises at most or about 200 μL of whole blood. In some embodiments, the sample comprises at most or about 150 μL of whole blood. In some embodiments, the sample comprises at most or about 100 μL of whole blood. In some embodiments, the sample comprises at most or about 50 μL of whole blood. In some embodiments, the sample comprises at most or about 400 μL of whole blood. In some embodiments, the sample comprises at most or about 30 μL of whole blood.

In some instances, the range of sample volumes is about 5 μl to about one milliliter. In some instances, the range of sample volumes is about 5 μl to about 900 μl. In some instances, the range of sample volumes is about 5 μl to about 800 μl. In some instances, the range of sample volumes is about 5 μl to about 700 μl. In some instances, the range of sample volumes is about 5 μl to about 600 μl. In some instances, the range of sample volumes is about 5 μl to about 500 μl. In some instances, the range of sample volumes is about 5 μl to about 400 μl. In some instances, the range of sample volumes is about 5 μl to about 300 μl. In some instances, the range of sample volumes is about 5 μl to about 200 μl. In some instances, the range of sample volumes is about 5 μl to about 150 μl. In some instances, the range of sample volumes is 5 μl to about 100 μl. In some instances, the range of sample volumes is about 5 μl to about 90 μl. In some instances, the range of sample volumes is about 5 μl to about 85 μl. In some instances, the range of sample volumes is about 5 μl to about 80 μl. In some instances, the range of sample volumes is about 5 μl to about 75 μl. In some instances, the range of sample volumes is about 5 μl to about 70 μl. In some instances, the range of sample volumes is about 5 μl to about 65 μl. In some instances, the range of sample volumes is about 5 μl to about 60 μl. In some instances, the range of sample volumes is about 5 μl to about 55 μl. In some instances, the range of sample volumes is about 5 μl to about 50 μl. In some instances, the range of sample volumes is about 15 μl to about 150 μl. In some instances, the range of sample volumes is about 15 μl to about 120 μl. In some instances, the range of sample volumes is 15 μl to about 100 μl. In some instances, the range of sample volumes is about 15 μl to about 90 μl. In some instances, the range of sample volumes is about 15 μl to about 85 μl. In some instances, the range of sample volumes is about 15 μl to about 80 μl. In some instances, the range of sample volumes is about 15 μl to about 75 μl. In some instances, the range of sample volumes is about 15 μl to about 70 μl. In some instances, the range of sample volumes is about 15 μl to about 65 μl. In some instances, the range of sample volumes is about 15 μl to about 60 μl. In some instances, the range of sample volumes is about 15 μl to about 55 μl. In some instances, the range of sample volumes is about 15 μl to about 50 μl.

The samples collected, in some cases comprises an ultra-low concentration of target analyte. In some cases, the target analytes are cell-free nucleic acids. The sample may comprise less than about 10¹⁰ cell-free nucleic acids. The sample may comprise about 10⁵ to about 10¹⁰ cell-free nucleic acids. The sample may comprise about 10⁴ to about 10¹⁰ cell-free nucleic acids. The sample may comprise about 10³ to about 10¹⁰ cell-free nucleic acids. The sample may comprise about 10² to about 10¹⁰ cell-free nucleic acids. The sample may comprise about 10⁵ to about 10⁹ cell-free nucleic acids. The sample may comprise about 10⁵ to about 10⁸ cell-free nucleic acids. The sample may comprise about 10⁵ to about 10⁷ cell-free nucleic acids. The sample may comprise about 10⁶ to about 10¹¹ cell-free nucleic acids. The sample may comprise about 10⁶ to about 10⁹ cell-free DNA. The sample may comprise about 10⁷ to about 10⁹ cell-free nucleic acids.

In some instances, the ultra-low amount is between about 4 pg to about 100 pg. In some instances, the ultra-low amount is between about 4 pg to about 150 pg. In some instances, the ultra-low amount is between about 4 pg to about 200 pg. In some instances, the ultra-low amount is between about 4 pg to about 300 pg. In some instances, the ultra-low amount is between about 4 pg to about 400 pg. In some instances, the ultra-low amount is between about 4 pg to about 500 pg. In some instances, the ultra-low amount is between about 4 pg to about 1 ng. In some instances, the ultra-low amount is between about 10 pg to about 100 pg. In some instances, the ultra-low amount is between about 10 pg to about 150 pg. In some instances, the ultra-low amount is between about 10 pg to about 200 pg. In some instances, the ultra-low amount is between about 10 pg to about 300 pg. In some instances, the ultra-low amount is between about 10 pg to about 400 pg. In some instances, the ultra-low amount is between about 10 pg to about 500 pg. In some instances, the ultra-low amount is between about 10 pg to about 1 ng. In some instances, the ultra-low amount is between about 20 pg to about 100 pg. In some instances, the ultra-low amount is between about 20 pg to about 200 pg. In some instances, the ultra-low amount is between about 20 pg to about 500 pg. In some instances, the ultra-low amount is between about 20 pg to about 1 ng. In some instances, the ultra-low amount is between about 30 pg to about 150 pg. In some instances, the ultra-low amount is between about 30 pg to about 180 pg. In some instances, the ultra-low amount is between about 30 pg to about 200 pg. In some instances, the ultra-low amount is between is about 30 pg to about 300 pg. In some instances, the ultra-low amount is between about 30 pg to about 400 pg. In some instances, the ultra-low amount is between about 30 pg to about 500 pg. In some instances, the ultra-low amount is between is about 30 pg to about 1 ng. In some instance, the subject is a pregnant subject and the cell-free nucleic acids comprise cell-free fetal DNA. In some instances, the subject has a tumor and the cell-free nucleic acids comprise cell-free tumor DNA. In some instances, the subject is an organ transplant recipient and the cell-free nucleic acids comprise organ donor DNA.

In embodiments, the sampling device receives an arbitrary liquid sample input containing the molecular analyte of interest. In some embodiment, the analyte of interest is a biomolecule such as DNA, RNA, protein, peptide, metabolite, lipid, virion, cell, or conjugate or aggregation of entities in from classes. In embodiments, the analyte is an organic or inorganic molecule that interacts with biological systems, such as a toxin, carcinogen, teratogen, stimulant, psychotropic, depressant, immunosuppressants, or pharmaceutical. In some embodiments, the sample is of biological origin, such as blood, saliva, urine, mucus, tissue secretions, or fecal liquid. In other embodiments, the sample is of synthetic origin, such as biochemical buffers, secretions from bioengineered cells, pharmacological precursors, or matrix solution for cultured tissue cells. In other embodiments, the sample is of environmental origin, such as runoff from vegetation, streams, rivers, ponds, lakes, aquifers, or oceans. In an embodiment, the sample may be the byproduct of infrastructure and process industries such as agricultural runoff, food processing and packaging, solvent purification, and sanitation systems. In an embodiment, a liquid sample is a byproduct of processing a solid or gas containing the molecular analyte of interest. For example, the sample may be diluted breath condensate, lysed tissue collected from biopsy, liquified bone matter, soil dilution, melted agar solutions, or a resuspended lyophilized product.

In some embodiments, sampling devices and systems disclosed herein comprise a sample collector. In some embodiments, the sample collector is provided separately from the rest of the device, system or kit. In some embodiments, the sample collector is physically integrated with the device, system or kit, or a component thereof. In some embodiments, the sample collector is integrated with a receptacle described herein. In some embodiments, the sample collector can be a cup, tube, capillary, or well for applying the biological fluid. In some embodiments, the sample collector can be a cup for applying urine. In some embodiments, the sample collector can comprise a pipet for applying urine in the cup to the device, system or kit. In some embodiments, the sample collector can be a capillary integrated with a device disclosed herein for applying blood. In some embodiments, the sample collector can be tube, well, pad or paper integrated with a device disclosed herein for applying saliva. In some embodiments, the sample collector can be pad or paper for applying sweat.

In some embodiments, sampling devices and systems disclosed herein comprise a transdermal puncture device. Non-limiting examples of transdermal puncture devices are needles and lancets. In some embodiments, the sample collector comprises the transdermal puncture device. In some embodiments, sampling devices and systems disclosed herein comprise a microneedle, microneedle array or microneedle patch. In some embodiments, sampling devices and systems disclosed herein comprise a hollow microneedle. By way of non-limiting example, the transdermal puncture device is integrated with a well or capillary so that as the subject punctures their finger, blood is released into the well or capillary where it will be available to the system or device for analysis of its components. In some embodiments, the transdermal puncture device is a push button device with a needle or lancet in a concave surface. In some embodiments, the needle is a microneedle. In some embodiments, the transdermal puncture device comprises an array of microneedles. By pressing an actuator, button or location on the non-needle side of the concave surface, the needle punctures the skin of the subject in a more controlled manner than a lancet. Furthermore, the push button device can comprise a vacuum source or plunger to help draw blood from the puncture site.

### Sample Processing and Purification

The sampling devices and systems described herein, in some cases, comprise a sample processor, wherein the sample processor modifies a biological sample to remove a component of the sample or separate the sample into multiple fractions (e.g., blood cell fraction and plasma or serum). The sample processor can comprise a sample purifier, wherein the sample purifier is configured to remove an unwanted substance or non-target component of a biological sample, thereby modifying the sample. Depending on the source of the biological sample, unwanted substances can include, but are not limited to, proteins (e.g., antibodies, hormones, enzymes, serum albumin, lipoproteins), free amino acids and other metabolites, microvesicles, nucleic acids, lipids, electrolytes, urea, urobilin, pharmaceutical drugs, mucous, bacteria, and other microorganisms, and combinations thereof. In some embodiments, the sample purifier separates components of a biological sample disclosed herein. In some embodiments, sample purifiers disclosed herein remove components of a sample that would inhibit, interfere with or otherwise be detrimental to the later process steps such as nucleic acid amplification or detection. In some embodiments, the resulting modified sample is enriched for target analytes. This can be considered indirect enrichment of target analytes. Alternatively or additionally, target analytes can be captured directly, which is considered direct enrichment of target analytes.

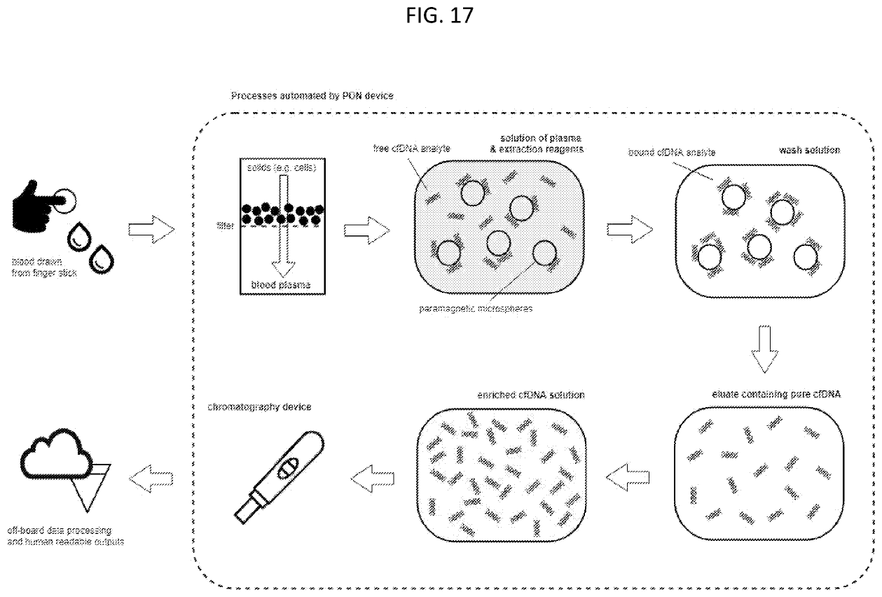

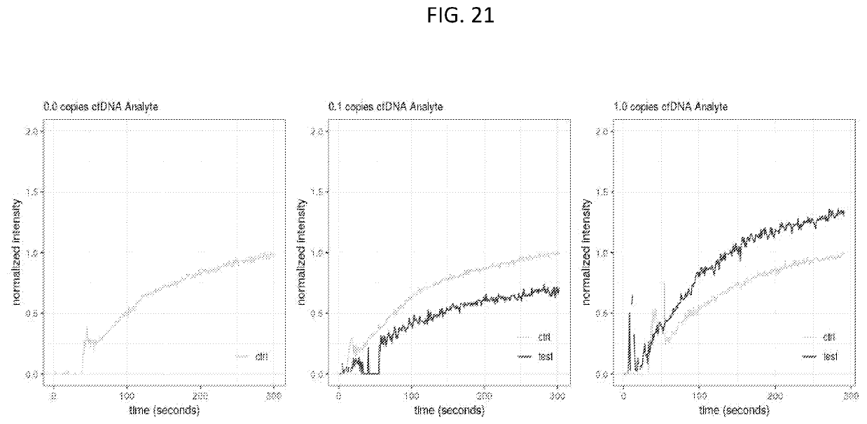

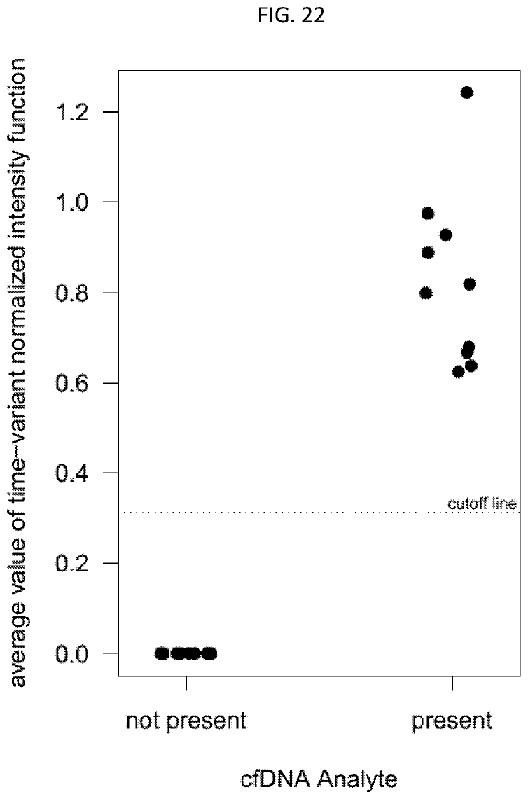

According to an embodiment, a specific example process executed by a sampling device of the kind previously described in FIG. 16 is provided in FIG. 17. In the embodiment, the device receives an input of a droplet of blood extracted from a fingertip and produces output signals correlating to its levels of cell free DNA (“cfDNA”) analyte. The droplet of blood is first filtered of solid matter, including red blood cells, white blood cells, apoptotic bodies, and viral particles. The resulting blood plasma is then combined with an aqueous solution of salts, paramagnetic microspheres, polymer surfactants, and buffers. cfDNA molecules bind noncovalently to the surface of the microspheres over a period of time. The microspheres are separated from the solution, washed further with aqueous solutions, and then exposed to an aqueous solvent that acts to elute purified cfDNA from their surfaces. The eluate may contain as little as 1 molecule of cfDNA, making it difficult to detect by conventional means. The eluate is then mixed with a solution of salts, polymer surfactants, and enzymes. This solution is heated, causing the number of cfDNA molecules to grow over a period of time. The resultant solution enriched with synthetic copies of cfDNA analyte is then diluted in an aqueous buffer and introduced to a chromatographic paper strip. The enriched solution travels down the strip, creating an optically detectable signal for both sample and controls. An output dataset from a unit of the invention for such a process is provided in FIG. 21. The time-variant output normalized signal intensity from a chromatographic strip in the form of a lateral flow assays shows the development of flow control and sample signals for multiple analyte concentration controls. The performance across a number of such devices on human blood samples is provided in FIG. 22 by further processing time-variant output data.

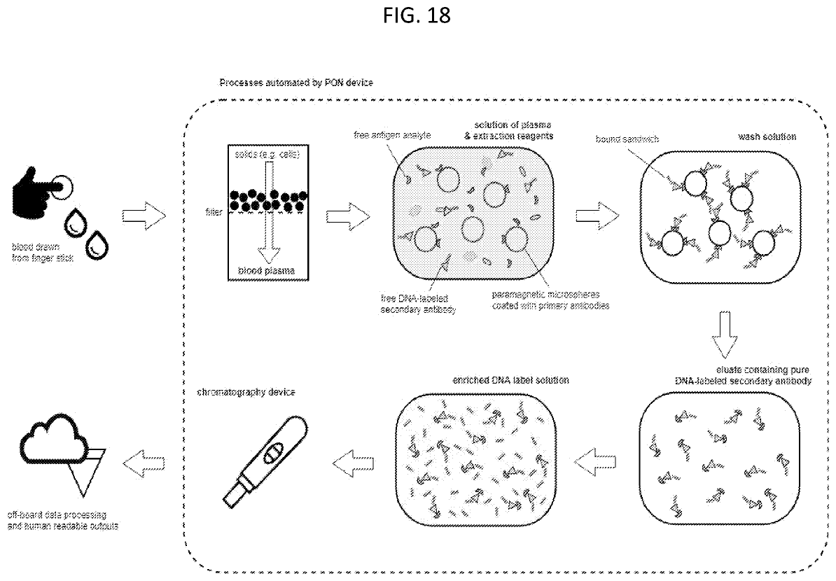

According to another embodiment, a specific example process executed by a sampling device of the kind previously described in FIG. 16 is provided in FIG. 18. In the embodiment, the sampling device receives an input of a droplet of blood extracted from a fingertip and produces output signals correlating to its levels of an antigen analyte. The droplet of blood is first filtered of solid matter, including red blood cells, white blood cells, apoptotic bodies, and viral particles. The resulting blood plasma is then combined with an aqueous solution of salts, paramagnetic microspheres, antibodies, polymer surfactants, and buffers. The microspheres are previously coated with a primary antibody that selectively binds the antigen analyte with high affinity. However, these primary antibodies may also bind analyte homologues and other proteins in solution nonspecifically. The solution also contains secondary antibodies that selectively bind a different epitope of the antigen analyte with high affinity. These secondary antibodies are previously conjugated to a DNA molecular label that can later be detected, creating greater specificity in the test when compared to detecting the antigen directly. In this manner, proteins bind to the microspheres, creating a “sandwich” or “bound triad” composed of primary antibodies, antigens, and DNA-labeled secondary antibodies. The microspheres are separated from the solution, washed further with aqueous solutions, and then exposed to an aqueous solvent that acts to elute the DNA-labeled secondary antibody from their surfaces. The quantity of DNA-labeled secondary antibody is proportional to the quantity of analyte antigen and therefore can be used to detect it. The eluate may contain as little as 1 molecule of DNA-labeled secondary antibody, making it difficult to detect by conventional means. The eluate is then mixed with a solution of salts, polymer surfactants, and enzymes. This solution is heated, causing the number of DNA molecules to grow over a period of time from the label conjugated to the secondary antibody, creating a relatively amplified endpoint detectable signal. The resultant solution enriched with synthetic copies of DNA is then diluted in an aqueous buffer and introduced to a chromatographic paper strip. The enriched solution travels down the strip, creating an optically detectable signal for both sample and controls.

In some embodiments, the sample purifier comprises a separation material for removing unwanted substances other than patient cells from the biological sample. Useful separation materials can include specific binding moieties that bind to or associate with the substance. Binding can be covalent or noncovalent. Any suitable binding moiety known in the art for removing a particular substance can be used. For example, antibodies and fragments thereof are commonly used for protein removal from samples. In some embodiments, a sample purifier disclosed herein comprises a binding moiety that binds a nucleic acid, protein, cell surface marker, or microvesicle surface marker in the biological sample. In some embodiments, the binding moiety comprises an antibody, antigen binding antibody fragment, a ligand, a receptor, a peptide, a small molecule, or a combination thereof.

In some embodiments, sample purifiers disclosed herein comprise a filter. In some embodiments, sample purifiers disclosed herein comprise a membrane. Generally, the filter or membrane is capable of separating or removing cells, cell particles, cell fragments, blood components other than cell-free nucleic acids, or a combination thereof, from the biological samples disclosed herein.

In some embodiments, the sample purifier facilitates separation of plasma or serum from cellular components of a blood sample. In some embodiments, the sample purifier facilitates separation of plasma or serum from cellular components of a blood sample before starting a molecular amplification reaction or a sequencing reaction. Plasma or serum separation can be achieved by several different methods such as centrifugation, sedimentation or filtration. In some embodiments, the sample purifier comprises a filter matrix for receiving whole blood, the filter matrix having a pore size that is prohibitive for cells to pass through, while plasma or serum can pass through the filter matrix uninhibited. In some embodiments, the filter matrix combines a large pore size at the top with a small pore size at the bottom of the filter, which leads to very gentle treatment of the cells preventing cell degradation or lysis, during the filtration process. This is advantageous because cell degradation or lysis would result in release of nucleic acids from blood cells or maternal cells that would contaminate target cell-free nucleic acids. Non-limiting examples of such filters include Pall Vivid™ GR membrane, Munktell Ahlstrom filter paper (see, e.g., WO 2017017314), TeraPore filters.

In some embodiments sampling devices and systems disclosed herein employ vertical filtration, driven by capillary force to separate a component or fraction from a sample (e.g., plasma from blood). By way of non-limiting example, vertical filtration can comprise gravitation assisted plasma separation. A high-efficiency superhydrophobic plasma separator is described, e.g., by Liu et al., A High Efficiency Superhydrophobic Plasma Separation, Lab Chip 2015.

The sample purifier can comprise a lateral filter (e.g., sample does not move in a gravitational direction or the sample moves perpendicular to a gravitational direction). The sample purifier can comprise a vertical filter (e.g., sample moves in a gravitational direction). The sample purifier can comprise vertical filter and a lateral filter. The sample purifier can be configured to receive a sample or portion thereof with a vertical filter, followed by a lateral filter. The sample purifier can be configured to receive a sample or portion thereof with a lateral filter, followed by a vertical filter. In some embodiments, a vertical filter comprises a filter matrix. In some embodiments, the filter matrix of the vertical filter comprises a pore with a pore size that is prohibitive for cells to pass through, while plasma can pass the filter matrix uninhibited. In some embodiments, the filter matrix comprises a membrane that is especially suited for this application because it combines a large pore size at the top with a small pore size at the bottom of the filter, which leads to very gentle treatment of the cells preventing cell degradation during the filtration process.

In some embodiments, the sample purifier comprises an appropriate separation material, e.g., a filter or membrane, which removes unwanted substances from a biological sample without removing cell-free nucleic acids. In some embodiments, the separation material separates substances in the biological sample based on size, for example, the separation material has a pore size that excludes a cell but is permeable to cell-free nucleic acids. Therefore, when the biological sample is blood, the plasma or serum can move more rapidly than a blood cell through the separation material in the sample purifier, and the plasma or serum containing any cell-free nucleic acids permeates the holes of the separation material. In some embodiments, the biological sample is blood, and the cell that is slowed and/or trapped in the separation material is a red blood cell, a white blood cell, or a platelet. In some embodiments, the cell is from a tissue that contacted the biological sample in the body, including, but not limited to, a bladder or urinary tract epithelial cell (in urine), or a buccal cell (in saliva). In some embodiments, the cell is a bacterium or other microorganism.

In some embodiments, the sample purifier is capable of slowing and/or trapping a cell without damaging the cell, thereby avoiding the release of cell contents including cellular nucleic acids and other proteins or cell fragments that could interfere with subsequent evaluation of the cell-free nucleic acids. This can be accomplished, for example, by a gradual, progressive reduction in pore size along the path of a lateral flow strip or other suitable assay format, to allow gentle slowing of cell movement, and thereby minimize the force on the cell. In some embodiments, at least 95%, at least 98%, at least 99%, or up to 100% of the cells in a biological sample remain intact when trapped in the separation material. In addition to or independently of size separation, the separation material can trap or separate unwanted substances based on a cell property other than size, for example, the separation material can comprise a binding moiety that binds to a cell surface marker. In some embodiments, the binding moiety is an antibody or antigen binding antibody fragment. In some embodiments, the binding moiety is a ligand or receptor binding protein for a receptor on a blood cell or microvesicle.

In some embodiments, systems and devices disclosed herein comprise a separation material that moves, draws, pushes, or pulls the biological sample through the sample purifier, filter and/or membrane. In some embodiments, the material is a wicking material. Examples of appropriate separation materials used in the sample purifier to remove cells include, but are not limited to, polyvinylidene difluoride, polytetrafluoroethylene, acetylcellulose, nitrocellulose, polycarbonate, polyethylene terephthalate, polyethylene, polypropylene, glass fiber, borosilicate, vinyl chloride, silver. Suitable separation materials can be characterized as preventing passage of cells. In some embodiments, the separation material is not limited as long as it has a property that can prevent passage of the red blood cells. In some embodiments, the separation material is a hydrophobic filter, for example a glass fiber filter, a composite filter, for example Cytosep (e.g., Ahlstrom Filtration or Pall Specialty Materials, Port Washington, NY), or a hydrophilic filter, for example cellulose (e.g., Pall Specialty Materials). In some embodiments, whole blood can be fractionated into red blood cells, white blood cells and serum components for further processing according to the methods of the present disclosure using a commercially available kit (e.g., Arrayit Blood Card Serum Isolation Kit, Cat. ABCS, Arrayit Corporation, Sunnyvale, Calif.).

In some embodiments the sample purifier comprises at least one filter or at least one membrane characterized by at least one pore size. In some embodiments, the sample purifier comprises multiple filters and/or membranes, wherein the pore size of at least a first filter or membrane differs from a second filter or membrane. In some embodiments, at least one pore size of at least one filter/membrane is about 0.05 microns to about 10 microns. In some embodiments, the pore size is about 0.05 microns to about 8 microns. In some embodiments, the pore size is about 0.05 microns to about 6 microns. In some embodiments, the pore size is about 0.05 microns to about 4 microns. In some embodiments, the pore size is about 0.05 microns to about 2 microns. In some embodiments, the pore size is about 0.05 microns to about 1 micron. In some embodiments, at least one pore size of at least one filter/membrane is about 0.1 microns to about 10 microns. In some embodiments, the pore size is about 0.1 microns to about 8 microns. In some embodiments, the pore size is about 0.1 microns to about 6 microns. In some embodiments, the pore size is about 0.1 microns to about 4 microns. In some embodiments, the pore size is about 0.1 microns to about 2 microns. In some embodiments, the pore size is about 0.1 microns to about 1 micron.

In some embodiments, the sample purifier is characterized as a gentle sample purifier. Gentle sample purifiers, such as those comprising a filter matrix, a vertical filter, a wicking material, or a membrane with pores that do not allow passage of cells, are particularly useful for analyzing cell-free nucleic acids. For example, prenatal applications of cell-free fetal nucleic acids in maternal blood are presented with the additional challenge of analyzing cell-free fetal nucleic acids in the presence of cell-free maternal nucleic acids, the latter of which create a large background signal to the former. By way of non-limiting example, a sample of maternal blood can contain about 500 to 750 genome equivalents of total cell-free DNA (maternal and fetal) per milliliter of whole blood when the sample is obtained without cell lysis or other cell disruption caused by the sample collection method. The fetal fraction in blood sampled from pregnant women can be around 10%, about 50 to 75 genome equivalents per ml. The process of obtaining cell-free nucleic acids usually involves obtaining plasma from the blood. If not performed carefully, maternal white blood cells can be destroyed, releasing additional cellular nucleic acids into the sample, creating a lot of background noise to the fetal cell-free nucleic acids. The typical white cell count is around 4*10\hat{φ}6 to 10*10\hat{ }6 cells per ml of blood and therefore the available nuclear DNA is around 4,000 to 10,000 times higher than the overall cell-free DNA (cfDNA). Consequently, even if only a small fraction of maternal white blood cells is destroyed, releasing nuclear DNA into the plasma, the fetal fraction is reduced dramatically. For example, a white cell degradation of 0.01% can reduce the fetal fraction from 10% to about 5%. Sampling devices and systems disclosed herein aim to reduce these background signals.

In some embodiments, the sample processor is configured to separate blood cells from whole blood. In some embodiments, the sample processor is configured to isolate plasma from whole blood. In some embodiments, the sample processor is configured to isolate serum from whole blood. In some embodiments, the sample processor is configured to isolate plasma or serum from less than 1 milliliter of whole blood. In some embodiments, the sample processor is configured to isolate plasma or serum from less than 1 milliliter of whole blood. In some embodiments, the sample processor is configured to isolate plasma or serum from less than 500 μL of whole blood. In some embodiments, the sample processor is configured to isolate plasma or serum from less than 400 μL of whole blood. In some embodiments, the sample processor is configured to isolate plasma or serum from less than 300 μL of whole blood. In some embodiments, the sample processor is configured to isolate plasma or serum from less than 200 μL of whole blood. In some embodiments, the sample processor is configured to isolate plasma or serum from less than 150 μL of whole blood. In some embodiments, the sample processor is configured to isolate plasma or serum from less than 100 μL of whole blood.

In some embodiments, the biological sample comprises fetal trophoblasts, that in some cases, contain the genetic information of a fetus (e.g., RNA, DNA). In some embodiments, fetal trophoblasts are enriched in the biological sample, such as by using an antibody against a fetal cell-surface antigen of morphology (e.g., size, shape). In some embodiments, the fetal trophoblasts are (1) isolated from the biological sample; (2) the isolated trophoblasts are lysed; (3) the fetal nuclei from the lysed fetal trophoblasts are isolated; (4) lysing the isolated fetal nuclei; and (5) purifying the genomic DNA from the isolated fetal nuclei.

In some embodiments, sampling devices and systems disclosed herein comprise a binding moiety for producing a modified sample depleted of cells, cell fragments, nucleic acids or proteins that are unwanted or of no interest. In some embodiments, sampling devices and systems disclosed herein comprise a binding moiety for reducing cells, cell fragments, nucleic acids or proteins that are unwanted or of no interest, in a biological sample. In some embodiments, sampling devices and systems disclosed herein comprise a binding moiety for producing a modified sample enriched with target cell, target cell fragments, target nucleic acids or target proteins.

In some embodiments, sampling devices and systems disclosed herein comprise a binding moiety capable of binding a nucleic acid, a protein, a peptide, a cell surface marker, or microvesicle surface marker. In some embodiments, sampling devices and systems disclosed herein comprise a binding moiety for capturing an extracellular vesicle or extracellular microparticle in the biological sample. In some embodiments, the extracellular vesicle contains at least one of DNA and RNA. In some embodiments, sampling devices and systems disclosed herein comprise reagents or components for analyzing DNA or RNA contained in the extracellular vesicle. In some embodiments, the binding moiety comprises an antibody, antigen binding antibody fragment, a ligand, a receptor, a protein, a peptide, a small molecule, or a combination thereof.

In some embodiments, sampling devices and systems disclosed herein comprise a binding moiety capable of interacting with or capturing an extracellular vesicle that is released from a cell. In some embodiments, the cell is a fetal cell. In some embodiments, the cell is a placental cell. The fetal cell or the placental cell can be circulating in a biological fluid (e.g., blood) of a female pregnant subject. In some embodiments, the extracellular vesicle is released from an organ, gland or tissue. By way of non-limiting example, the organ, gland or tissue can be diseased, aging, infected, or growing. Non-limiting examples of organs, glands and tissues are brain, liver, heart, kidney, colon, pancreas, muscle, adipose, thyroid, prostate, breast tissue, and bone marrow.

By way of non-limiting example, sampling devices and systems disclosed herein can be capable of capturing and discarding an extracellular vesicle or extracellular microparticle from a maternal sample to enrich the sample for fetal/ placental nucleic acids. In some embodiments, the extracellular vesicle is fetal/ placental in origin. In some embodiments, the extracellular vesicle originates from a fetal cell. In some embodiments, the extracellular vesicle is released by a fetal cell. In some embodiments, the extracellular vesicle is released by a placental cell. The placental cell can be a trophoblast cell. In some embodiments, sampling devices and systems disclosed herein comprise a cell-binding moiety for capturing placenta educated platelets, which can contain fetal DNA or RNA fragments. These can be captured/ enriched for with antibodies or other methods (low speed centrifugation). In such embodiments, the fetal DNA or RNA fragments can be analyzed as described herein to detect or indicate chromosomal information (e.g., gender). Alternatively or additionally, sampling devices and systems disclosed herein comprise a binding moiety for capturing an extracellular vesicle or extracellular microparticle in the biological sample that comes from a maternal cell.

In some embodiments, the binding moiety is attached to a solid support, wherein the solid support can be separated from the rest of the biological sample or the biological sample can be separated from the solid support, after the binding moiety has made contact with the biological sample. Non-limiting examples of solid supports include a bead, a nanoparticle, a magnetic particle, a chip, a microchip, a fibrous strip, a polymer strip, a membrane, a matrix, a column, a plate, or a combination thereof.

Sampling devices and systems disclosed herein can comprise a cell lysis reagent. Non-limiting examples of cell lysis reagents include detergents such as NP-40, sodium dodecyl sulfate, and salt solutions comprising ammonium, chloride, or potassium. Sampling devices and systems disclosed herein can have a cell lysis component. The cell lysis component can be structural or mechanical and capable of lysing a cell. By way of non-limiting example, the cell lysis component can shear the cells to release intracellular components such as nucleic acids. In some embodiments, sampling devices and systems disclosed herein do not comprise a cell lysis reagent. Some sampling devices and systems disclosed herein are intended to analyze cell-free nucleic acids.

### Molecular Analyte Detection

Disclosed herein, in some embodiments, are systems and devices (e.g., sampling devices) that detect one or more molecular analytes. An “analyte detector” of the devices and systems can, in some cases, perform the analyte detection described herein. In some embodiments, the sampling device produces one or more detectable signals related to the presence of one or more molecular analytes in an input sample or other liquids stored on-board. Example signals include “negative controls” indicating absence of analyte, “positive controls” indicating the presence of analyte, “quantification controls” to allow for quantitative measurements against reference material, “flow controls” indicating presence or absence of liquid, “standard values” proportional to known quantities of analyte, and “test values” indicating the presence, absence, or quantity of analyte in a sample.

In an embodiment, a detected signal is qualitative, indicating the presence or absence of an analyte in a sample. In another embodiment, a detected signal is quantitative, varying proportional to the amount or concentration of an analyte in a sample. In this case, the amount or concentration of an analyte is discerned by comparing its associated signal to standard values. The standard values may be produced by processing a set of calibration reagents. The calibration reagents may be stored within the invention itself or used externally as an input to the invention in place of a sample. Alternatively, the standard values may be stored in the invention's local memory, accessed over a network connection to the invention, or encoded on the invention's packaging such that paired network devices can later decode this information. Example formats in which standard values may be encoded on the invention's packaging are QR codes, barcodes, and RFID tags. In an embodiment, a detected signal is semi-quantitative, indicating the amount of an analyte relative to a single or range of reference values or thresholds. Reference values may be standard values, control values, or arbitrary values of interest. In one embodiment, reference values are defined based on a statistical analysis of measured signal responses across a population of blood samples originating from different persons or from similar persons at different points of time.

In an embodiment, detected signals are collected in a time variant manner. In another embodiment, detected signals are collected in a static manner relative to an arbitrary point in time. Additional processing of a time variant signal may be executed by electronics and software onboard the sampling device or through a device to which it is networked in order to create a new output signal or data. Example processing includes computing the signal's rate of change, root-mean-square power, fourier transform, nonlinear regression, and classification by neural network.

In an embodiment, detected output signals are generated from the detection of one or more analytes in a single sampling device unit. In an embodiment, the signals are generated in a multiplexed format or generated simultaneously from a liquid input in a single detection module. In another embodiment, the signals are generated in a parallelized format, or generated simultaneously from aliquoted liquid inputs in multiple detection modules. In another embodiment, signals are generated in a sequential format, or generated from aliquoted liquid inputs in a single detection module such that only one aliquot is in the detection module at any given time.

In some embodiments, sampling devices and systems disclosed herein comprise at least one of a nucleic acid detector, capture component, signal detector, a detection reagent, or a combination thereof, for detecting a nucleic acid in the biological sample. In some embodiments, the capture component and the signal detector are integrated. In some embodiments, the capture component comprises a solid support. In some embodiments the solid support comprises a bead, a chip, a strip, a membrane, a matrix, a column, a plate, or a combination thereof.

The systems and devices (e.g., sampling devices) disclosed herein, in some cases, detect molecular analytes that are nucleic acids. In some embodiments, sampling devices and systems disclosed herein comprise at least one probe for an epigenetically modified region of a chromosome or fragment thereof. In some embodiments, the epigenetic modification of the epigenetically modified region of a chromosome is indicative of gender or a marker of gender. In some embodiments, sampling devices and systems disclosed herein comprise at least one probe for a paternally inherited sequence that is not present in the maternal DNA. In some embodiments, sampling devices and systems disclosed herein comprise at least one probe for a paternally inherited single nucleotide polymorphism. In some embodiments, the chromosome is a Y chromosome. In some embodiments, the chromosome is an X chromosome. In some embodiments, the chromosome is a Y chromosome. In some embodiments, the chromosome is an autosome. In some embodiments, the probe comprises a peptide, an antibody, an antigen binding antibody fragment, a nucleic acid or a small molecule.

In some embodiments, sampling devices and systems comprise a sample purifier disclosed herein, and a capture component disclosed herein. In some embodiments, the sample purifier comprises the capture component. In some embodiments, the sample purifier and the capture component are integrated. In some embodiments, the sample purifier and the capture component are separate.

In some embodiments, the capture component comprises a binding moiety described herein. In some embodiments, the binding moiety is present in a lateral flow assay. In some embodiments, the binding moiety is added to the sample before the sample is added to the lateral flow assay. In some embodiments, the binding moiety comprises a signaling molecule. In some embodiments, the binding moiety is physically associated with a signaling molecule. In some embodiments, the binding moiety is capable of physically associating with a signaling molecule. In some embodiments, the binding moiety is connected to a signaling molecule. Non-limiting examples of signaling molecules include a gold particle, a fluorescent particle, a luminescent particle, and a dye molecule. In some embodiments the capture component comprises a binding moiety that is capable of interacting with an amplification product described herein. In some embodiments the capture component comprises a binding moiety that is capable of interacting with a tag on an amplification product described herein.

In some embodiments, sampling devices and systems disclosed herein comprise a detection system. In some embodiments, the detection system comprises a signal detector. Non-limiting examples of a signal detector include a fluorescence reader, a colorimeter, a sensor, a wire, a circuit, a receiver. In some embodiments, the detection system comprises a detection reagent. Non-limiting examples of a detection reagent include a fluorophore, a chemical, a nanoparticle, an antibody, and a nucleic acid probe. In some embodiments, the detection system comprises a pH sensor and a complementary metal-oxide semiconductor, which can be used to detect changes in pH. In some embodiments, production of an amplification product by devices, systems, kits or methods disclosed herein changes the pH, thereby indicating genetic information.

In some embodiments, the system comprises a signal detector. In some embodiments, the signal detector is a photodetector that detects photons. In some embodiments, the signal detector detects fluorescence. In some embodiments, the signal detector detects a chemical or compound. In some embodiments, the signal detector detects a chemical that is released when the amplification product is produced. In some embodiments, the signal detector detects a chemical that is released when the amplification product is added to the detection system. In some embodiments, the signal detector detects a compound that is produced when the amplification product is produced. In some embodiments, the signal detector detects a compound that is produced when the amplification product is added to the detection system.

In some embodiments, the signal detector detects an electrical signal. In some embodiments, the signal detector comprises an electrode. In some embodiments, the signal detector comprises a circuit a current, or a current generator. In some embodiments, the circuit or current is provided by a gradient of two or more solutions or polymers. In some embodiments, the circuit or current is provided by an energy source (e.g., battery, cell phone, wire from electrical outlet). In some embodiments, nucleic acids, amplification products, chemicals or compounds disclosed herein provide an electrical signal by disrupting the current and the signal detector detects the electrical signal.

In some embodiments, the signal detector detects light. In some embodiments, the signal detector comprises a light sensor. In some embodiments, the signal detector comprises a camera. In some embodiments, the signal detector comprises a cell phone camera or a component thereof.

In some embodiments, the signal detector comprises a nanowire that detects the charge of different bases in nucleic acids. In some embodiments, the nanowire has a diameter of about 1 nm to about 99 nm. In some embodiments, the nanowire has a diameter of about 1 nm to about 999 nm. In some embodiments, the nanowire comprises an inorganic molecule, e.g., nickel, platinum, silicon, gold, zinc, graphene, or titanium. In some embodiments, the nanowire comprises an organic molecule (e.g., a nucleotide).

In some embodiments, the devices and systems comprise an assay assembly, wherein the assay assembly is capable of detecting a target analyte (e.g., nucleic acid amplification product). In some embodiments, the assay assembly comprises a lateral flow strip, also referred to herein and in the field, as a lateral flow assay, lateral flow test or lateral flow device. In some embodiments, a lateral flow assay provides a fast, inexpensive, and technically simple method to detect amplification products disclosed herein. Generally, lateral flow assays disclosed herein comprise a porous material or porous matrix that transports a fluid, and a detector that detects the amplification product when it is present. The porous material can comprise a porous paper, a polymer structure, a sintered polymer, or a combination thereof. In some embodiments, the lateral flow assay transports the biological fluid or portion thereof (e.g., plasma of blood sample). In some embodiments, the lateral flow assay transports a solution containing the biological fluid or portion thereof. For instance, methods can comprise adding a solution to the biological fluid before or during addition of the sample to the device or system. The solution can comprise a salt, a polymer, or any other component that facilitates transport of the sample and or amplification product through the lateral flow assay. In some embodiments, nucleic acids are amplified after they have traveled through the lateral flow strip.

In some embodiments, the devices and systems comprise a lateral flow device, wherein the lateral flow device comprises multiple sectors or zones, wherein each desired function can be present in a separate sector or zone. In general, in a lateral flow device, a liquid sample, e.g., a body fluid sample as described herein, containing the target analyte moves with or without the assistance of external forces through sectors or zones of the lateral flow device. In some embodiments, the target analyte moves without the assistance of external forces, e.g., by capillary action. In some embodiments, the target analyte moves with assistance of external forces, e.g., by facilitation of capillary action by movement of the lateral flow device. Movement can comprise any motion caused by external input, e.g., shaking, turning, centrifuging, applying an electrical field or magnetic field, applying a pump, applying a vacuum, or rocking of the lateral flow device.

In some embodiments, the lateral flow device is a lateral flow test strip, comprising zones or sectors that are situated laterally, e.g., behind or ahead of each other. In general, a lateral flow test strip allows accessibility of the functional zones or sectors from each side of (e.g., above and below) the test strip as a result of exposure of a large surface area of each functional zone or sector. This facilitates the addition of reagents, including those used in sample purification, or target analyte amplification, and/or detection.

Any suitable lateral flow test strip detection format known to those of skill in the art is contemplated for use in an assay assembly of the present disclosure. Lateral flow test strip detection formats are well known and have been described in the literature. Lateral flow test strip assay formats are generally described by, e.g., Sharma et al., (2015) Biosensors 5:577-601, incorporated by reference herein in its entirety. Detection of nucleic acids using lateral flow test strip sandwich assay formats is described by, e.g., U.S. Pat. No. 9,121,849, “Lateral Flow Assays,” incorporated by reference herein in its entirety. Detection of nucleic acids using lateral flow test strip competitive assay formats is described by, e.g., U.S. Pat. No. 9,423,399, “Lateral Flow Assays for Tagged Analytes,” incorporated by reference herein in its entirety.

In some embodiments, a lateral flow test strip detects the target analyte in a test sample using a sandwich format, a competitive format, or a multiplex detection format. In a traditional sandwich assay format, the detected signal is directly proportional to the amount of the target analyte present in the sample, so that increasing amounts of the target analyte lead to increasing signal intensity. In traditional competitive assay formats, the detected signal has an inverse relationship with the amount of analyte present and increasing amounts of analyte lead to decreasing signal intensity.

In a lateral flow sandwich format, also referred to as a “sandwich assay,” the test sample typically is applied to a sample application pad at one end of a test strip. The applied test sample flows through the test strip, from the sample application pad to a conjugate pad located adjacent to the sample application pad, where the conjugate pad is downstream in the direction of sample flow. In some embodiments, the conjugate pad comprises a labeled, reversibly-immobilized probe, e.g., an antibody or aptamer labeled with, e.g., a dye, enzyme, or nanoparticle. A labeled probe-target analyte complex is formed if the target analyte is present in the test sample. This complex then flows to a first test zone or sector (e.g., a test line) comprising an immobilized second probe which is specific to the target analyte, thereby trapping any labeled probe-target analyte complex. In some embodiments, the intensity or magnitude of signal, e.g., color, at the first test zone or sector is used to indicate the presence or absence, quantity, or presence and quantity of target analyte in the test sample. A second test zone or sector can comprise a third probe that binds to excess labeled probe. If the applied test sample comprises the target analyte, little or no excess labeled probe will be present on the test strip following capture of the target analyte by the labeled probe on the conjugate pad. Consequently, the second test zone or sector will not bind any labeled probe, and little or no signal (e.g., color) at the second test zone or sector is expected to be observed. The absence of signal at the second test zone or sector thus can provide assurance that signal observed in the first test zone or sector is due to the presence of the target analyte.

In some embodiments, sampling devices and systems disclosed herein comprise a sandwich assay. In some embodiments, the sandwich assay is configured to receive a biological sample disclosed herein and retain sample components (e.g., nucleic acids, cells, microparticles). In some embodiments, the sandwich assay is configured to receive a flow solution that flushes non-nucleic acid components of the biological sample (e.g., proteins, cells, microparticles), leaving nucleic acids of the biological sample behind. In some embodiments, the sandwich assay comprises a membrane that binds nucleic acids to help retain the nucleic acids when the flow solution is applied. Non-limiting examples of a membrane the binds nucleic acids include chitosan modified nitrocellulose.

Similarly, in a lateral flow competitive format a test sample is applied to a sample application pad at one end of a test strip, and the target analyte binds to a labeled probe to form a probe-target analyte complex in a conjugate pad downstream of the sample application pad. In the competitive format, the first test zone or sector typically comprises the target analyte or an analog of the target analyte. The target analyte in the first test zone or sector binds any free labeled probe that did not bind to the test analyte in the conjugate pad. Thus, the amount of signal observed in the first test zone or sector is higher when there is no target analyte in the applied test sample than when target analyte is present. A second test zone or sector comprises a probe that specifically binds to the probe-target analyte complex. The amount of signal observed in this second test zone or sector is higher when the target analyte is present in the applied test sample.

In a lateral flow test strip multiplex detection format, more than one target analyte is detected using the test strip through the use of additional test zones or sectors comprising, e.g., probes specific for each of the target analytes.

In some embodiments, the lateral flow device is a layered lateral flow device, comprising zones or sectors that are present in layers situated medially, e.g., above or below each other. In some embodiments, one or more zones or sectors are present in a given layer. In some embodiments, each zone or sector is present in an individual layer. In some embodiments, a layer comprises multiple zones or sectors. In some embodiments, the layers are laminated. In a layered lateral flow device, processes controlled by diffusion and directed by the concentration gradient are possible driving forces. For example, multilayer analytical elements for fluorometric assay or fluorometric quantitative analysis of an analyte contained in a sample liquid are described in EP0097952, “Multilayer analytical element,” incorporated by reference herein.

A lateral flow device can comprise one or more functional zones or sectors. In some embodiments, the test assembly comprises 1 to 20 functional zones or sectors. In some embodiments, the functional zones ore sectors comprise at least one sample purification zone or sector, at least one target analyte amplification zone or sector, at least one target analyte detection zone or sector, and at least one target analyte detection zone or sector.

In some embodiments, the target analyte is a nucleic acid sequence, and the lateral flow device is a nucleic acid lateral flow assay. In some embodiments, sampling devices and systems disclosed herein comprise a nucleic acid lateral flow assay, wherein the nucleic acid lateral flow assay comprises nucleic acid amplification function. In some embodiments, target nucleic acid amplification that is carried out by the nucleic acid amplification function takes place prior to, or at the same time as, detection of the amplified nucleic acid species. In some embodiments, detection comprises one or more of qualitative, semi-quantitative, or quantitative detection of the presence of the target analyte.

In some embodiments, sampling devices and systems disclosed herein comprise an assay assembly wherein a target nucleic acid analyte is amplified in a lateral flow test strip to generate a labeled amplification product, or an amplification product that can be labeled after amplification. In some embodiments, a label is present on one or more amplification primers, or subsequently conjugated to one or more amplification primers, following amplification. In some embodiments, at least one target nucleic acid amplification product is detected on the lateral flow test strip. For example, one or more zones or sectors on the lateral flow test strip can comprise a probe that is specific for a target nucleic acid amplification product.

In some embodiments, the sampling devices and systems disclosed herein comprise a detector, wherein the detector comprises a graphene biosensor. Graphene biosensors are described, e.g., by Afsahi et al., in the article entitled, “Novel graphene-based biosensor for early detection of Zika virus infection, Biosensor and Bioelectronics,” (2018) 100:85-88.

In some embodiments, a detector disclosed herein comprises a nanopore, a nanosensor, or a nanoswitch. For instance, the detector can be capable of nanopore sequencing, a method of transporting a nucleic acid through a nanopore based on an electric current across a membrane, the detector measuring disruptions in the current corresponding to specific nucleotides. A nanoswitch or nanosensor undergoes a structural change upon exposure to the detectable signal. See, e.g., Koussa et al., “DNA nanoswitches: A quantitative platform for gel-based biomolecular interaction analysis,” (2015) Nature Methods, 12(2): 123-126.

In some embodiments, the detector comprises a rapid multiplex biomarker assay where probes for an analyte of interest are produced on a chip that is used for real-time detection. Thus, there is no need for a tag, label or reporter. Binding of analytes to these probes causes a change in a refractive index that corresponds to a concentration of the analyte. All steps can be automated. Incubations can be not be necessary. Results can be available in less than an hour (e.g., 10-30 minutes). A non-limiting example of such a detector is the Genalyte Maverick Detection System.

### Additional Tests

In some embodiments, sampling devices and systems disclosed herein comprise additional features, reagents, tests or assays for detection or analysis of biological components besides nucleic acids. By way of non-limiting example, the biological component can be selected from a peptide, a lipid, a fatty acid, a sterol, a carbohydrate, a viral component, a microbial component, and a combination thereof. The biological component can be an antibody. The biological component can be an antibody produced in response to a peptide in the subject. These additional assays can be capable of detecting or analyzing biological components in the small volumes or sample sizes disclosed herein and throughout. An additional test can comprise a reagent capable of interacting with a biological component of interest. Non-limiting examples of such reagents include antibodies, peptides, oligonucleotides, aptamers, and small molecules, and combinations thereof. The reagent can comprise a detectable label. The reagent can be capable of interacting with a detectable label. The reagent can be capable of providing a detectable signal.

Additional tests can require one or more antibodies. For instance, the additional test can comprise reagents or components that provide for performing Immuno-PCR (IPCR). IPCR is a method wherein a first antibody for a protein of interest is immobilized and exposed to a sample. If the sample contains the protein of interest, it will be captured by the first antibody. The captured protein of interest is then exposed to a second antibody that binds the protein of interest. The second antibody has been coupled to a polynucleotide that can be detected by real-time PCR. Alternatively or additionally, the additional test can comprise reagents or components that provide for performing a proximity ligation assay (PLA), wherein the sample is exposed to two antibodies specific for a protein of interest, each antibody comprising an oligonucleotide. If both antibodies bind to the protein of interest, the oligonucleotides of each antibody will be close enough to be amplified and/or detected.

In some embodiments, sampling devices and systems disclosed herein comprise a pregnancy test to confirm the subject is pregnant. In some embodiments, sampling devices and systems disclosed herein comprise a test for presence of a Y chromosome or absence of a Y chromosome (gender test). In some embodiments, sampling devices and systems disclosed herein comprise a test for gestational age.

In some embodiments, sampling devices and systems disclosed herein comprise a test for multiple pregnancies, e.g., twins or triplets. In some embodiments, methods disclosed herein quantify (absolute or relative) the total amount of fetal nucleic acids in a maternal sample, and the amount of sequences represented by the various autosomes, X and Y chromosomes to detect if one, both or all fetuses are male or female, euploid or aneuploid, etc.

In some embodiments, sampling devices and systems disclosed herein comprise a pregnancy test for indicating, detecting or verifying the subject is pregnant. In some embodiments the pregnancy test comprises a reagent or component for measuring a pregnancy related factor. By way of non-limiting example, the pregnancy related factor can be human chorionic gonadotropin protein (hCG) and the reagent or component for hCG comprising an anti-hCG antibody. Also by way of non-limiting example, the pregnancy related factor can be an hCG transcript and the reagent or component for measuring the hCG transcript is an oligonucleotide probe or primer that hybridizes to the hCG transcript. In some embodiments, the pregnancy related factor is heat shock protein 10 kDa protein 1, also known as early-pregnancy factor (EPF).

In some embodiments, sampling devices and systems disclosed herein are capable of conveying the age of the fetus. For example, a signal can be generated from the device or system, wherein the level of the signal corresponds to the amount of hCG in the sample from the subject. This level or strength of the signal can be translated or equivocated with a numerical value representing the amount of hCG in the sample. The amount of hCG can indicate an approximate age of the fetus.

In some embodiments, sampling devices and systems disclosed herein provide an indication or verification of pregnancy, an indication or verification of gestational age, and an indication or verification of gender. In some embodiments, sampling devices and systems disclosed herein provide an indication of pregnancy, gestational age, and/or gender with at least about 90% confidence (e.g., 90% of the time, the indication is accurate). In some embodiments, sampling devices and systems disclosed herein provide an indication of pregnancy, gestational age, and/or gender with at least about 95% confidence. In some embodiments, sampling devices and systems disclosed herein provide an indication of pregnancy, gestational age, and/or gender with at least about 99% confidence.

Disclosed herein, in some embodiments are devices and systems comprising a nucleic acid detector that can detect a target nucleic acid. In some embodiments, the nucleic acid detector comprises a nucleic acid sequencer. In some embodiments, sampling devices and systems disclosed herein are configured to amplify nucleic acids and sequence the resulting amplified nucleic acids. In some embodiments, sampling devices and systems disclosed herein are configured to sequence nucleic acids without amplifying nucleic acids. In some embodiments, sampling devices and systems disclosed herein comprise a nucleic acid sequencer, but do not comprise a nucleic acid amplifying reagent or nucleic acid amplifying component. In some embodiments, the nucleic acid sequencer comprises a signal detector that detects a signal that reflects successful amplification or unsuccessful amplification. In some embodiments, the nucleic acid sequencer is the signal detector. In some embodiments, the signal detector comprises the nucleic acid sequencer.

In some embodiments, the nucleic acid sequencer has a communication connection with an electronic device that analyzes sequencing reads from the nucleic acid sequencer. In some embodiments the communication connection is hard wired. In some embodiments the communication connection is wireless. For example, a mobile device app or computer software, such as those disclosed herein, can receive the sequencing reads, and based on the sequencing reads, display or report genetic information about the sample (e.g., presence of a disease/infection, response to a drug, genetic abnormality or mutation of a fetus).

In some embodiments, the nucleic acid sequencer comprises a nanopore sequencer. In some embodiments, the nanopore sequencer comprises a nanopore. In some embodiments, the nanopore sequencer comprises a membrane and solutions that create a current across the membrane and drive movement of charged molecules (e.g., nucleic acids) through the nanopore. In some embodiments, the nanopore sequencer comprises a transmembrane protein, a portion thereof, or a modification thereof. In some embodiments, the transmembrane protein is a bacterial protein. In some embodiments, the transmembrane protein is not a bacterial protein. In some embodiments, the nanopore is synthetic. In some embodiments, the nanopore performs solid state nanopore sequencing. In some embodiments, the nanopore sequencer is described as pocket-sized, portable, or roughly the size of a cell phone. In some embodiments, the nanopore sequencer is configured to sequence at least one of RNA and DNA. Non-limiting examples of nanopore sequencing devices include Oxford Nanopore Technologies MinION and SmidgION nanopore sequencing USB devices. Both of these devices are small enough to be handheld. Nanopore sequencing devices and components are further described in reviews by Howorka (Nat Nanotechnol. 2017 July 6;12(7):619-630), and Garrido-Cardenas et al. (Sensors (Basel). 2017 March 14;17(3)), both incorporated herein by reference. Other non-limiting examples of nanopore sequencing devices are offered by Electronic Biosciences, Two Pore Guys, Stratos, and Agilent (technology originally from Genia).

In some embodiments, the nucleic acid detector comprises reagents and components required for bisulfite sequencing to detect epigenetic modifications. For instance, a long region with many methylation markers can be fragmented. Here, each fragment carrying a methylation marker can be an independent signal. Signals from all the fragments are sufficient in combination to obtain useful genetic information.

In some embodiments, the nucleic acid detector does not comprise a nucleic acid sequencer. In some embodiments, the nucleic acid detector is configured to count tagged nucleic acids, wherein the nucleic acid detector quantifies a collective signal from one or more tags.

The systems and devices (e.g., sampling devices) disclosed herein, in some cases, detect analytes that are polypeptides or proteins. In some embodiments, a “polypeptide detector” of the systems and devices disclosed herein is configured for polypeptide or protein detection.

In some embodiments, the sampling devices and systems disclosed herein utilize optically based, electrochemical, electro-optical and other methods that leverage the enzyme-linked immunosorbent assays (ELISAs) approach to analyte detection. In some embodiments, protein arrays that employ optical or electrical detection are utilized by the sampling devices and systems disclosed herein. In some embodiments, the protein array provides a selective protein capture agent on a chip. In some embodiments, the protein array may contain primary antibodies or aptamers to capture the desired analyte polypeptides or proteins, and after washing with a cocktail designed to block non-specific binding, a labeled secondary antibody dispersion is added to bind to the analyte proteins. In some cases, the labeled secondary antibody can provide an optical or electrical signal. In some cases, the labeled second antibody comprises an enzyme label.

### Analyte Enrichment

Generally, devices (e.g., sampling devices) and systems disclosed herein can enrich an analyte in a sample. In some embodiments, the devices and systems disclosed herein comprise an “enricher,” configured to enrich or increase a concentration of the analyte in a sample. An enricher is configured to enrich the analyte in coordination with the analyte detector, to optimize the sensitivity of the device or system.

A. Nucleic Acid Amplification

Disclosed herein are devices and systems comprising an enricher configured to amplify or enrich a target nucleic acid in a sample. Often sampling devices and systems disclosed herein comprise a DNA polymerase. In some embodiments, the sampling devices and systems disclosed herein comprise a reverse transcriptase enzyme to produce complementary DNA (cDNA) from RNA in biological samples disclosed herein, wherein the cDNA can be amplified and/or analyzed similarly to genomic DNA as described herein. Sampling devices and systems disclosed herein also often contain a crowding agent which can increase the efficiency enzymes like DNA polymerases and helicases. Crowding agents can increase an efficiency of a library, as described elsewhere herein. The crowding agent can comprise a polymer, a protein, a polysaccharide, or a combination thereof. Non-limiting examples of crowding agents that can be used in sampling devices and systems disclosed herein are dextran, poly (ethylene glycol) and dextran.

A traditional polymerase chain reaction requires thermocycling. This would be possible, but inconvenient for a typical at-home user without a thermocycler machine. In some embodiments, sampling devices and systems disclosed herein are capable of amplifying a nucleic acid without changing the temperature of the device or system or a component thereof. In some embodiments, sampling devices and systems disclosed herein are capable of amplifying a nucleic acid isothermally. Non-limiting examples of isothermal amplification are as follows: loop-mediated isothermal amplification (LAMP), strand displacement amplification (SDA), helicase dependent amplification (HDA), nicking enzyme amplification reaction (NEAR), and recombinase polymerase amplification (RPA). Thus, sampling devices and systems disclosed herein can comprise reagents necessary to carry out an isothermal amplification. Non-limiting examples of isothermal amplification reagents include recombinase polymerases, single-strand DNA-binding proteins, and strand-displacing polymerases. Generally, isothermal amplification using recombinase polymerase amplification (RPA) employs three core enzymes, recombinase, single-strand DNA-binding protein, and strand-displacing polymerase, to (1) pair oligonucleotide primers with homologous sequence in DNA, (2) stabilize displaced DNA strands to prevent primer displacement, and (3) extend the oligonucleotide primer using a strand displacing DNA polymerase. Using paired oligonucleotide primers, exponential DNA amplification can take place with incubation at room temperature (optimal at 37° C.).

In some embodiments, sampling devices and systems disclosed herein are capable of amplifying a nucleic acid at a temperature. In some embodiments, sampling devices and systems disclosed herein are capable of amplifying a nucleic acid at not more than two temperatures. In some embodiments, sampling devices and systems disclosed herein are capable of amplifying a nucleic acid at not more than three temperatures. In some embodiments, sampling devices and systems disclosed herein only require initially heating one reagent or component of the device, system or kit.

In some embodiments, sampling devices and systems disclosed herein are capable of amplifying a nucleic acid at a range of temperatures. In some embodiments, the range of temperatures is about −50° C. to about 100° C. In some embodiments, the range of temperatures is about −50° C. to about 90° C. In some embodiments, the range of temperatures is about −50° C. to about 80° C. In some embodiments, the range of temperatures is about is about −50° C. to about 70° C. In some embodiments, the range of temperatures is about −50° C. to about 60° C. In some embodiments, the range of temperatures is about −50° C. to about 50° C. In some embodiments, the range of temperatures is about −50° C. to about 40° C. In some embodiments, the range of temperatures is about −50° C. to about 30° C. In some embodiments, the range of temperatures is about −50° C. to about 20° C. In some embodiments, the range of temperatures is about −50° C. to about 10° C. In some embodiments, the range of temperatures is about 0° C. to about 100° C. In some embodiments, the range of temperatures is about 0° C. to about 90° C. In some embodiments, the range of temperatures is about 0° C. to about 80° C. In some embodiments, the range of temperatures is about is about 0° C. to about 70° C. In some embodiments, the range of temperatures is about 0° C. to about 60° C. In some embodiments, the range of temperatures is about 0° C. to about 50° C. In some embodiments, the range of temperatures is about 0° C. to about 40° C. In some embodiments, the range of temperatures is about 0° C. to about 30° C. In some embodiments, the range of temperatures is about 0° C. to about 20° C. In some embodiments, the range of temperatures is about 0° C. to about 10° C. In some embodiments, the range of temperatures is about 15° C. to about 100° C. In some embodiments, the range of temperatures is about 15° C. to about 90° C. In some embodiments, the range of temperatures is about 15° C. to about 80° C. In some embodiments, the range of temperatures is about is about 15° C. to about 70 ° C. In some embodiments, the range of temperatures is about 15° C. to about 60° C. In some embodiments, the range of temperatures is about 15° C. to about 50° C. In some embodiments, the range of temperatures is about 15° C. to about 40° C. In some embodiments, the range of temperatures is about 15° C. to about 30° C. In some embodiments, the range of temperatures is about 10° C. to about 30° C. In some embodiments, devices, systems, kits disclosed herein, including all components thereof, and all reagents thereof, are completely operable at room temperature, not requiring cooling, freezing or heating.

In some embodiments, at least a portion of the sampling devices and systems disclosed herein operate at about 20° C. to about 50° C. In some embodiments, at least a portion of the sampling devices and systems disclosed herein operate at about 37° C. In some embodiments, at least a portion of the sampling devices and systems disclosed herein operate at about 42° C. In some embodiments, the sampling devices and systems disclosed herein are advantageously operated at room temperature. In some embodiments, at least a portion of the sampling devices and systems disclosed herein are capable of amplifying a nucleic acid isothermally at about 20° C. to about 30° C. In some embodiments, at least a portion of the sampling devices and systems disclosed herein are capable of amplifying a nucleic acid isothermally at about 23° C. to about 27° C.

In some embodiments, sampling devices and systems disclosed herein comprise a hybridization probe with an abasic site, a fluorophore and quencher to monitor amplification. Exonuclease III can be included to cleave the abasic site and release the quencher to allow fluorescent excitation. In some embodiments, amplification products are detected or monitored via lateral flow by attaching a capture molecule (e.g. Biotin) to one of the amplification primers and labeling a hybridization primer with a 5′-antigenic molecule (e.g. fluorescein derivative FAM) for capture to allow for detection. As such, in some embodiments, sampling devices and systems disclosed herein provide for detection of nucleic acids and amplification products on a lateral flow device. Lateral flow devices are described herein.

In some embodiments, sampling devices and systems disclosed herein comprise at least one nucleic acid amplification reagent and at least one oligonucleotide primer capable of amplifying a first sequence in a genome and a second sequence in a genome, wherein the first sequence and the second sequence are similar, and wherein the first sequence is physically distant enough from the second sequence such that the first sequence is present on a first cell-free nucleic acid of the subject and the second sequence is present on a second cell-free nucleic acid of the subject. In some embodiments, the at least two sequences are immediately adjacent. In some embodiments the at least two sequences are separated by at least one nucleotide. In some embodiments, the at least two sequences are separated by at least two nucleotides. In some embodiments, the at least two sequences are separated by at least about 5, at least about 10, at least about 15, at least about 20, at least about 30, at least about 40, at least about 50, or at least about 100 nucleotides. In some embodiments, the at least two sequences are at least about 50% identical. In some embodiments, the at least two sequences are at least about 60% identical, at least about 60% identical, at least about 60%, at least about 70%, at least about 80%, at least about 90%, at least about 95%, at least about 99%, or 100% identical. In some embodiments, the first sequence and the second sequence are each at least 10 nucleotides in length. In some embodiments, the first sequence and the second sequence are each at least about 10, at least about 15, at least about 20, at least about 30, at least about 50, or at least about 100 nucleotides in length. In some embodiments, the first sequence and the second sequence are on the same chromosome. In some embodiments, the first sequence is on a first chromosome and the second sequence is on a second chromosome. In some embodiments, the first sequence and the second sequence are in functional linkage. For example, all CpG sites in the promotor region of gene AOX1 show the same hypermethylation in prostate cancer, so these sites are in functional linkage because they functionally carry the same information but are located one or more nucleotides apart.

In some embodiments, sampling devices and systems disclosed herein comprise at least one of an oligonucleotide probe or oligonucleotide primer that is capable of annealing to a strand of a cell-free nucleic acid, wherein the cell-free nucleic acid comprises a sequence corresponding to a region of interest or a portion thereof. In some embodiments, the region of interest is a region of a Y chromosome. In some embodiments, the region of interest is a region of an X chromosome. In some embodiments, the region of interest is a region of an autosome. In some embodiments, the region of interest, or portion thereof, comprises a repeat sequence as described herein that is present in a genome more than once. In some embodiments, the region of interest is about 10 nucleotides to about 1,000,000 nucleotides in length. In some embodiments, the region of interest is at least 10 nucleotides in length. In some embodiments, the region of interest is at least 100 nucleotides in length. In some embodiments, the region is at least 1000 nucleotides in length. In some embodiments, the region of interest is about 10 nucleotides to about 500,000 nucleotides in length. In some embodiments, the region of interest is about 10 nucleotides to about 300,000 nucleotides in length. In some embodiments, the region of interest is about 100 nucleotides to about 1,000,000 nucleotides in length. In some embodiments, the region of interest is about 100 nucleotides to about 500,000 nucleotides in length. In some embodiments, the region of interest is about 100 nucleotides to about 300,000 base pairs in length. In some embodiments, the region of interest is about 1000 nucleotides to about 1,000,000 nucleotides in length. In some embodiments, the region of interest is about 1000 nucleotides to about 500,000 nucleotides in length. In some embodiments, the region of interest is about 1000 nucleotides to about 300,000 nucleotides in length. In some embodiments, the region of interest is about 10,000 nucleotides to about 1,000,000 nucleotides in length. In some embodiments, the region of interest is about 10,000 nucleotides to about 500,000 nucleotides in length. In some embodiments, the region of interest is about 10,000 nucleotides to about 300,000 nucleotides in length. In some embodiments, the region of interest is about 300,000 nucleotides in length.

In some embodiments, the sequence corresponding to the region of interest is at least about 5 nucleotides in length. In some embodiments, the sequence corresponding to the region of interest is at least about 8 nucleotides in length. In some embodiments, the sequence corresponding to the region of interest is at least about 10 nucleotides in length. In some embodiments, the sequence corresponding to the region of interest is at least about 15 nucleotides in length. In some embodiments, the sequence corresponding to the region of interest is at least about 20 nucleotides in length. In some embodiments, the sequence corresponding to the region of interest is at least about 50 nucleotides in length. In some embodiments, the sequence corresponding to the region of interest is at least about 100 nucleotides in length. In some embodiments, the sequence is about 5 nucleotides to about 1000 nucleotides in length. In some embodiments, the sequence is about 10 nucleotides to about 1000 nucleotides in length. In some embodiments, the sequence is about 10 nucleotides to about 500 nucleotides in length. In some embodiments, the sequence is about 10 nucleotides to about 400 nucleotides in length. In some embodiments, the sequence is about 10 nucleotides to about 300 nucleotides in length. In some embodiments, the sequence is about 50 nucleotides to about 1000 nucleotides in length. In some embodiments, the sequence is about 50 nucleotides to about 500 nucleotides in length.

In some embodiments, sampling devices and systems disclosed herein comprise at least one of an oligonucleotide probe and oligonucleotide primer that is capable of annealing to a strand of a cell-free nucleic acid, wherein the cell-free nucleic acid comprises a sequence corresponding to a sub-region of interest disclosed herein. In some embodiments, the sub-region is represented by a sequence that is present in the region of interest more than once. In some embodiments, the sub-region is about 10 to about 1000 nucleotides in length. In some embodiments, the sub-region is about 50 to about 500 nucleotides in length. In some embodiments, the sub-region is about 50 to about 250 nucleotides in length. In some embodiments, the sub-region is about 50 to about 150 nucleotides in length. In some embodiments, the sub-region is about 100 nucleotides in length.

In some embodiments, sampling devices and systems disclosed herein comprise at least one oligonucleotide primer, wherein the oligonucleotide primer has a sequence complementary to or corresponding to a Y chromosome sequence. In some embodiments, devices, systems and kits disclosed herein comprise a pair of oligonucleotide primers, wherein the pair of oligonucleotide primers have sequences complementary to or corresponding to a Y chromosome sequence. In some embodiments, devices, systems and kits disclosed herein comprise at least one oligonucleotide primer, wherein the oligonucleotide primer comprises a sequence complementary to or corresponding to a Y chromosome sequence. In some embodiments, devices, systems and kits disclosed herein comprise a pair of oligonucleotide primers, wherein the pair of oligonucleotide primers comprise sequences complementary to or corresponding to a Y chromosome sequence. In some embodiments, devices, systems and kits disclosed herein comprise at least one oligonucleotide primer, wherein the oligonucleotide primer consists of a sequence complementary to or corresponding to a Y chromosome sequence. In some embodiments, devices, systems and kits disclosed herein comprise a pair of oligonucleotide primers, wherein the pair of oligonucleotide primers consists of sequences complementary to or corresponding to a Y chromosome sequence. In some embodiments, the sequence(s) complementary to or corresponding to a Y chromosome sequence is at least 75% identical to a wild-type human Y chromosome sequence. In some embodiments, the sequence(s) complementary to or corresponding to a Y chromosome sequence is at least 80% identical to a wild-type human Y chromosome sequence. In some embodiments, the sequence(s) complementary to or corresponding to a Y chromosome sequence is at least 85% identical to a wild-type human Y chromosome sequence. In some embodiments, the sequence(s) complementary to or corresponding to a Y chromosome sequence is at least 80% identical to a wild-type human Y chromosome sequence. In some embodiments, the sequence(s) complementary to or corresponding to a Y chromosome sequence is at least 90% identical to a wild-type human Y chromosome sequence. In some embodiments, the sequence(s) complementary to or corresponding to a Y chromosome sequence is at least 95% identical to a wild-type human Y chromosome sequence. In some embodiments, the sequence(s) complementary to or corresponding to a Y chromosome sequence is at least 97% identical to a wild-type human Y chromosome sequence. In some embodiments, the sequence(s) complementary to or corresponding to a Y chromosome sequence is 100% identical to a wild-type human Y chromosome sequence.

In some embodiments, sampling devices and systems disclosed herein comprise at least one of an oligonucleotide probe and oligonucleotide primer that is capable of annealing to a strand of a cell-free nucleic acid, wherein the cell-free nucleic acid comprises a sequence corresponding to a Y chromosome region, or portion thereof, wherein the portion thereof has a given length. In some embodiments, the length of the portion thereof is about 10 nucleotides to about 100 nucleotides. In some embodiments, the length of the portion thereof is about 100 nucleotides to about 1000 nucleotides. In some embodiments, the length of the portion thereof is about 1000 nucleotides to about 10,000 nucleotides. In some embodiments, the length of the portion thereof is about 10,000 nucleotides to about 100,000 nucleotides.

In some embodiments, the region of interest is a Y chromosome region, or portion thereof, that comprises a sequence that is present on the Y chromosome more than once. In some embodiments, the Y chromosome region is located between position 20000000 and position 21000000 of the Y chromosome. In some embodiments, the Y chromosome region is located between position 20500000 and position 21000000 of the Y chromosome. In some embodiments, the Y chromosome region is located between position 20000000 and position 20500000 of the Y chromosome. In some embodiments, the Y chromosome region is located between position 20000000 and position 20250000 of the Y chromosome. In some embodiments, the Y chromosome region is located between position 20250000 and position 20500000 of the Y chromosome. In some embodiments, the Y chromosome region is located between position 20500000 and position 20750000 of the Y chromosome. In some embodiments, the Y chromosome region is located between position 20750000 and position 21000000 of the Y chromosome. In some embodiments, the Y chromosome region is located between position 20080000 and position 20400000 of the Y chromosome. In some embodiments, the Y chromosome region is located between position 20082000 and position 20351000 of the Y chromosome. In some embodiments, the Y chromosome region is located between position 20082183 and position 20350897of the Y chromosome.

In some embodiments, devices, systems and kits disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region. In some embodiments, corresponding is 100% identical. In some embodiments, corresponding is at least 99% identical. In some embodiments, corresponding is at least 98% identical. In some embodiments, corresponding is at least 95% identical. In some embodiments, corresponding is at least 90% identical.

In some embodiments, sampling devices and systems disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 20350799 and end position 20350897 of the Y chromosome. In some embodiments, the sequence corresponds to at least 10 nucleotides of a Y chromosome sub-region between start position 20350799 and end position 20350897 of the Y chromosome. In some embodiments, the sequence corresponds to at least 50 nucleotides of a Y chromosome sub-region between start position 20350799 and end position 20350897 of the Y chromosome. In some embodiments, the sequence corresponds to at least about 10 to at least about 1000 nucleotides of a Y chromosome sub-region between start position 20350799 and end position 20350897 of the Y chromosome. In some embodiments, the sequence corresponds to at least about 50 to at least about 500 nucleotides of a Y chromosome sub-region between start position 20350799 and end position 20350897 of the Y chromosome. In some embodiments, the sequence corresponds to at least about 50 to at least about 150 nucleotides of a Y chromosome sub-region between start position 20350799 and end position 20350897 of the Y chromosome.

In some embodiments, sampling devices and systems disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 20349236 and end position 20349318 of the Y chromosome. In some embodiments, the sequence corresponds to at least 10 nucleotides of a Y chromosome sub-region between start position 20349236 and end position 20349318 of the Y chromosome. In some embodiments, the sequence corresponds to at least 50 nucleotides of a Y chromosome sub-region between start position 20349236 and end position 20349318 of the Y chromosome. In some embodiments, the sequence corresponds to at least about 10 to at least about 1000 nucleotides of a Y chromosome sub-region between start position 20349236 and end position 20349318 of the Y chromosome. In some embodiments, the sequence corresponds to at least about 50 to at least about 500 nucleotides of a Y chromosome sub-region between start position 20349236 and end position 20349318 of the Y chromosome. In some embodiments, the sequence corresponds to at least about 50 to at least about 150 nucleotides of a Y chromosome sub-region between start position 20349236 and end position 20349318 of the Y chromosome.

In some embodiments, sampling devices and systems disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 20350231 and end position 20350323 of the Y chromosome. In some embodiments, the sequence corresponds to at least 10 nucleotides of a Y chromosome sub-region between start position 20350231 and end position 20350323 of the Y chromosome. In some embodiments, the sequence corresponds to at least 50 nucleotides of a Y chromosome sub-region between start position 20350231 and end position 20350323 of the Y chromosome. In some embodiments, the sequence corresponds to at least about 10 to at least about 1000 nucleotides of a Y chromosome sub-region between start position 20350231 and end position 20350323 of the Y chromosome. In some embodiments, the sequence corresponds to at least about 50 to at least about 500 nucleotides of a Y chromosome sub-region between start position 20350231 and end position 20350323 of the Y chromosome. In some embodiments, the sequence corresponds to at least about 50 to at least about 150 nucleotides of a Y chromosome sub-region between start position 20350231 and end position 20350323 of the Y chromosome.

In some embodiments, sampling devices and systems disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 20350601 and end position 20350699 of the Y chromosome. In some embodiments, the sequence corresponds to at least 10 nucleotides of a Y chromosome sub-region between start position 20350601 and end position 20350699 of the Y chromosome. In some embodiments, the sequence corresponds to at least 50 nucleotides of a Y chromosome sub-region between start position 20350601 and end position 20350699 of the Y chromosome. In some embodiments, the sequence corresponds to at least about 10 to at least about 1000 nucleotides of a Y chromosome sub-region between start position 20350601 and end position 20350699 of the Y chromosome. In some embodiments, the sequence corresponds to at least about 50 to at least about 500 nucleotides of a Y chromosome sub-region between start position 20350601 and end position 20350699 of the Y chromosome. In some embodiments, the sequence corresponds to at least about 50 to at least about 150 nucleotides of a Y chromosome sub-region between start position 20350601 and end position 20350699 of the Y chromosome.

In some embodiments, sampling devices and systems disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 20082183 and end position 20082281 of the Y chromosome. In some embodiments, the sequence corresponds to at least 10 nucleotides of a Y chromosome sub-region between start position 20082183 and end position 20082281 of the Y chromosome. In some embodiments, the sequence corresponds to at least 50 nucleotides of a Y chromosome sub-region between start position 20082183 and end position 20082281 of the Y chromosome. In some embodiments, the sequence corresponds to at least about 10 to at least about 1000 nucleotides of a Y chromosome sub-region between start position 20082183 and end position 20082281 of the Y chromosome. In some embodiments, the sequence corresponds to at least about 50 to at least about 500 nucleotides of a Y chromosome sub-region between start position 20082183 and end position 20082281 of the Y chromosome. In some embodiments, the sequence corresponds to at least about 50 to at least about 150 nucleotides of a Y chromosome sub-region between start position 20082183 and end position 20082281 of the Y chromosome.

In some embodiments, sampling devices and systems disclosed herein comprise at least one of an oligonucleotide probe and oligonucleotide primer that is capable of annealing to a strand of a cell-free nucleic acid, wherein the cell free nucleic acid comprises a sequence corresponding to a Y chromosome sub-region between start position 56673250 and end position 56771489 of the Y chromosome. In some embodiments, the sequence corresponds to at least 10 nucleotides of a Y chromosome sub-region between start position 56673250 and end position 56771489 of the Y chromosome. In some embodiments, the sequence corresponds to at least 50 nucleotides of a Y chromosome sub-region between start position 56673250 and end position 56771489 of the Y chromosome. In some embodiments, the sequence corresponds to at least about 10 to at least about 1000 nucleotides of a Y chromosome sub-region between start position 56673250 and end position 56771489 of the Y chromosome. In some embodiments, the sequence corresponds to at least about 50 to at least about 500 nucleotides of a Y chromosome sub-region between start position 56673250 and end position 56771489 of the Y chromosome. In some embodiments, the sequence corresponds to at least about 50 to at least about 150 nucleotides of a Y chromosome sub-region between start position 56673250 and end position 56771489 of the Y chromosome.

Any appropriate nucleic acid amplification method known in the art is contemplated for use in the devices and methods described herein. In some embodiments, isothermal amplification is used. In some embodiments, amplification is isothermal with the exception of an initial heating step before isothermal amplification begins. A number of isothermal amplification methods, each having different considerations and providing different advantages, are known in the art and have been discussed in the literature, e.g., by Zanoli and Spoto, 2013, “Isothermal Amplification Methods for the Detection of Nucleic Acids in Microfluidic Devices,” Biosensors 3: 18-43, and Fakruddin, et al., 2013, “Alternative Methods of Polymerase Chain Reaction (PCR),” Journal of Pharmacy and Bioallied Sciences 5(4): 245-252, each incorporated herein by reference in its entirety. In some embodiments, any appropriate isothermic amplification method is used. In some embodiments, the isothermic amplification method used is selected from: Loop Mediated Isothermal Amplification (LAMP); Nucleic Acid Sequence Based Amplification (NASBA); Multiple Displacement Amplification (MDA); Rolling Circle Amplification (RCA); Helicase Dependent Amplification (HDA); Strand Displacement Amplification (SDA); Nicking Enzyme Amplification Reaction (NEAR); Ramification Amplification Method (RAM); and Recombinase Polymerase Amplification (RPA).

In some embodiments, the amplification method used is LAMP (see, e.g., Notomi, et al., 2000, “Loop Mediated Isothermal Amplification” NAR 28(12): e63 i-vii, and U.S. Pat. No. 6,410,278, “Process for synthesizing nucleic acid” each incorporated by reference herein in its entirety). LAMP is a one-step amplification system using auto-cycling strand displacement deoxyribonucleic acid (DNA) synthesis. In some embodiments, LAMP is carried out at 60-65° C. for 45-60 min in the presence of a thermostable polymerase, e.g., Bacillus stearothermophilus (Bst) DNA polymerase I, deoxyribonucleotide triphosphate (dNTPs), specific primers and the target DNA template. In some embodiments, the template is RNA and a polymerase having both reverse transcriptase activity and strand displacement-type DNA polymerase activity, e.g., Bca DNA polymerase, is used, or a polymerase having reverse transcriptase activity is used for the reverse transcriptase step and a polymerase not having reverse transcriptase activity is used for the strand displacement-DNA synthesis step.

In some embodiments, the amplification reaction is carried out using LAMP, at about 55 ° C. to about 70° C. In some embodiments, the LAMP reaction is carried out at 55° C. or greater. In some embodiments, the LAMP reaction is carried out 70° C. or less. In some embodiments, the LAMP reaction is carried out at about 55° C. to about 57° C., about 55° C. to about 59° C., about 55° C. to about 60° C., about 55° C. to about 61° C., about 55° C. to about 62° C., about 55° C. to about 63° C., about 55° C. to about 64° C., about 55° C. to about 65° C., about 55° C. to about 66° C., about 55° C. to about 68° C., about 55° C. to about 70° C., about 57° C. to about 59° C., about 57° C. to about 60° C., about 57° C. to about 61° C., about 57° C. to about 62° C., about 57° C. to about 63° C., about 57° C. to about 64° C., about 57° C. to about 65° C., about 57° C. to about 66° C., about 57° C. to about 68° C., about 57° C. to about 70° C., about 59° C. to about 60° C., about 59° C. to about 61° C., about 59° C. to about 62° C., about 59° C. to about 63° C., about 59° C. to about 64° C., about 59° C. to about 65° C., about 59° C. to about 66° C., about 59° C. to about 68° C., about 59° C. to about 70° C., about 60° C. to about 61° C., about 60° C. to about 62° C., about 60° C. to about 63° C., about 60° C. to about 64° C., about 60° C. to about 65° C., about 60° C. to about 66° C., about 60° C. to about 68° C., about 60° C. to about 70° C., about 61° C. to about 62° C., about 61° C. to about 63° C., about 61° C. to about 64° C., about 61° C. to about 65° C., about 61° C. to about 66° C., about 61° C. to about 68° C., about 61° C. to about 70° C., about 62° C. to about 63° C., about 62° C. to about 64° C., about 62° C. to about 65° C., about 62° C. to about 66° C., about 62° C. to about 68° C., about 62° C. to about 70° C., about 63° C. to about 64° C., about 63° C. to about 65° C., about 63° C. to about 66° C., about 63° C. to about 68° C., about 63° C. to about 70° C., about 64° C. to about 65° C., about 64° C. to about 66° C., about 64° C. to about 68° C., about 64° C. to about 70° C., about 65° C. to about 66° C., about 65° C. to about 68° C., about 65° C. to about 70° C., about 66° C. to about 68° C., about 66° C. to about 70° C., or about 68° C. to about 70° C. In some embodiments, the LAMP reaction is carried out at about 55° C., about 57° C., about 59° C., about 60° C., about 61° C., about 62° C., about 63° C., about 64° C., about 65° C., about 66° C., about 68° C., or about 70° C.

In some embodiments, the amplification reaction is carried out using LAMP, for about 30 to about 90 minutes. In some embodiments, the LAMP reaction is carried out for at least about 30 minutes. In some embodiments, the LAMP reaction is carried out for at most about 90 minutes. In some embodiments, the LAMP reaction is carried out for about 30 minutes to about 35 minutes, about 30 minutes to about 40 minutes, about 30 minutes to about 45 minutes, about 30 minutes to about 50 minutes, about 30 minutes to about 55 minutes, about 30 minutes to about 60 minutes, about 30 minutes to about 65 minutes, about 30 minutes to about 70 minutes, about 30 minutes to about 75 minutes, about 30 minutes to about 80 minutes, about 30 minutes to about 90 minutes, about 35 minutes to about 40 minutes, about 35 minutes to about 45 minutes, about 35 minutes to about 50 minutes, about 35 minutes to about 55 minutes, about 35 minutes to about 60 minutes, about 35 minutes to about 65 minutes, about 35 minutes to about 70 minutes, about 35 minutes to about 75 minutes, about 35 minutes to about 80 minutes, about 35 minutes to about 90 minutes, about 40 minutes to about 45 minutes, about 40 minutes to about 50 minutes, about 40 minutes to about 55 minutes, about 40 minutes to about 60 minutes, about 40 minutes to about 65 minutes, about 40 minutes to about 70 minutes, about 40 minutes to about 75 minutes, about 40 minutes to about 80 minutes, about 40 minutes to about 90 minutes, about 45 minutes to about 50 minutes, about 45 minutes to about 55 minutes, about 45 minutes to about 60 minutes, about 45 minutes to about 65 minutes, about 45 minutes to about 70 minutes, about 45 minutes to about 75 minutes, about 45 minutes to about 80 minutes, about 45 minutes to about 90 minutes, about 50 minutes to about 55 minutes, about 50 minutes to about 60 minutes, about 50 minutes to about 65 minutes, about 50 minutes to about 70 minutes, about 50 minutes to about 75 minutes, about 50 minutes to about 80 minutes, about 50 minutes to about 90 minutes, about 55 minutes to about 60 minutes, about 55 minutes to about 65 minutes, about 55 minutes to about 70 minutes, about 55 minutes to about 75 minutes, about 55 minutes to about 80 minutes, about 55 minutes to about 90 minutes, about 60 minutes to about 65 minutes, about 60 minutes to about 70 minutes, about 60 minutes to about 75 minutes, about 60 minutes to about 80 minutes, about 60 minutes to about 90 minutes, about 65 minutes to about 70 minutes, about 65 minutes to about 75 minutes, about 65 minutes to about 80 minutes, about 65 minutes to about 90 minutes, about 70 minutes to about 75 minutes, about 70 minutes to about 80 minutes, about 70 minutes to about 90 minutes, about 75 minutes to about 80 minutes, about 75 minutes to about 90 minutes, or about 80 minutes to about 90 minutes. In some embodiments, the LAMP reaction is carried out for about 30 minutes, about 35 minutes, about 40 minutes, about 45 minutes, about 50 minutes, about 55 minutes, about 60 minutes, about 65 minutes, about 70 minutes, about 75 minutes, about 80 minutes, or about 90 minutes.

In some embodiments, the amplification method is Nucleic Acid Sequence Based Amplification (NASBA). NASBA (also known as 3SR, and transcription-mediated amplification) is an isothermal transcription-based RNA amplification system. Three enzymes (avian myeloblastosis virus reverse transcriptase, RNase H and T7 DNA dependent RNA polymerase) are used to generate single-stranded RNA. In certain cases, NASBA can be used to amplify DNA. The amplification reaction is performed at 41° C., maintaining constant temperature, typically for about 60 to about 90 minutes (see, e.g., Fakruddin, et al., 2012, “Nucleic Acid Sequence Based Amplification (NASBA) Prospects and Applications,” Int. J. of Life Science and Pharma Res. 2(1): L106-L121, incorporated by reference herein).

In some embodiments, the NASBA reaction is carried out at about 40° C. to about 42° C. In some embodiments, the NASBA reaction is carried out at 41° C. In some embodiments, the NASBA reaction is carried out at most at about 42° C. In some embodiments, the NASBA reaction is carried out at about 40° C. to about 41° C., about 40° C. to about 42° C., or about 41° C. to about 42° C. In some embodiments, the NASBA reaction is carried out at about 40° C., about 41° C., or about 42° C.

In some embodiments, the amplification reaction is carried out using NASBA, for about 45 to about 120 minutes. In some embodiments, the NASBA reaction is carried out for about 30 minutes to about 120 minutes. In some embodiments, the NASBA reaction is carried out for at least about 30 minutes. In some embodiments, the NASBA reaction is carried out for at most about 120 minutes. In some embodiments, the NASBA reaction is carried out for up to 180 minutes. In some embodiments, the NASBA reaction is carried out for about 30 minutes to about 45 minutes, about 30 minutes to about 60 minutes, about 30 minutes to about 65 minutes, about 30 minutes to about 70 minutes, about 30 minutes to about 75 minutes, about 30 minutes to about 80 minutes, about 30 minutes to about 85 minutes, about 30 minutes to about 90 minutes, about 30 minutes to about 95 minutes, about 30 minutes to about 100 minutes, about 30 minutes to about 120 minutes, about 45 minutes to about 60 minutes, about 45 minutes to about 65 minutes, about 45 minutes to about 70 minutes, about 45 minutes to about 75 minutes, about 45 minutes to about 80 minutes, about 45 minutes to about 85 minutes, about 45 minutes to about 90 minutes, about 45 minutes to about 95 minutes, about 45 minutes to about 100 minutes, about 45 minutes to about 120 minutes, about 60 minutes to about 65 minutes, about 60 minutes to about 70 minutes, about 60 minutes to about 75 minutes, about 60 minutes to about 80 minutes, about 60 minutes to about 85 minutes, about 60 minutes to about 90 minutes, about 60 minutes to about 95 minutes, about 60 minutes to about 100 minutes, about 60 minutes to about 120 minutes, about 65 minutes to about 70 minutes, about 65 minutes to about 75 minutes, about 65 minutes to about 80 minutes, about 65 minutes to about 85 minutes, about 65 minutes to about 90 minutes, about 65 minutes to about 95 minutes, about 65 minutes to about 100 minutes, about 65 minutes to about 120 minutes, about 70 minutes to about 75 minutes, about 70 minutes to about 80 minutes, about 70 minutes to about 85 minutes, about 70 minutes to about 90 minutes, about 70 minutes to about 95 minutes, about 70 minutes to about 100 minutes, about 70 minutes to about 120 minutes, about 75 minutes to about 80 minutes, about 75 minutes to about 85 minutes, about 75 minutes to about 90 minutes, about 75 minutes to about 95 minutes, about 75 minutes to about 100 minutes, about 75 minutes to about 120 minutes, about 80 minutes to about 85 minutes, about 80 minutes to about 90 minutes, about 80 minutes to about 95 minutes, about 80 minutes to about 100 minutes, about 80 minutes to about 120 minutes, about 85 minutes to about 90 minutes, about 85 minutes to about 95 minutes, about 85 minutes to about 100 minutes, about 85 minutes to about 120 minutes, about 90 minutes to about 95 minutes, about 90 minutes to about 100 minutes, about 90 minutes to about 120 minutes, about 95 minutes to about 100 minutes, about 95 minutes to about 120 minutes, or about 100 minutes to about 120 minutes. In some embodiments, the NASBA reaction is carried out for about 30 minutes, about 45 minutes, about 60 minutes, about 65 minutes, about 70 minutes, about 75 minutes, about 80 minutes, about 85 minutes, about 90 minutes, about 95 minutes, about 100 minutes, about 120 minutes, about 150 minutes, or about 180 minutes.

In some embodiments, the amplification method is Strand Displacement Amplification (SDA). SDA is an isothermal amplification method that uses four different primers. A primer containing a restriction site (a recognition sequence for HincII exonuclease) is annealed to the DNA template. An exonuclease-deficient fragment of Eschericia coli DNA polymerase 1 (exo-Klenow) elongates the primers. Each SDA cycle consists of (1) primer binding to a displaced target fragment, (2) extension of the primer/target complex by exo-Klenow, (3) nicking of the resultant hemiphosphothioate HincII site, (4) dissociation of HincII from the nicked site and (5) extension of the nick and displacement of the downstream strand by exo-Klenow.

In some embodiments, the amplification method is Multiple Displacement Amplification (MDA). The MDA is an isothermal, strand-displacing method based on the use of the highly processive and strand-displacing DNA polymerase from bacteriophage 029, in conjunction with modified random primers to amplify the entire genome with high fidelity. It has been developed to amplify all DNA in a sample from a very small amount of starting material. In MDA 029 DNA polymerase is incubated with dNTPs, random hexamers and denatured template DNA at 30° C. for 16 to18 hours and the enzyme must be inactivated at high temperature (65° C.) for 10 min. No repeated recycling is required, but a short initial denaturation step, the amplification step, and a final inactivation of the enzyme are needed.

In some embodiments, the amplification method is Rolling Circle Amplification (RCA). RCA is an isothermal nucleic acid amplification method which allows amplification of the probe DNA sequences by more than 109-fold at a single temperature, typically about 30° C. Numerous rounds of isothermal enzymatic synthesis are carried out by 029 DNA polymerase, which extends a circle-hybridized primer by continuously progressing around the circular DNA probe. In some embodiments, the amplification reaction is carried out using RCA, at about 28° C. to about 32° C.

In some embodiments, sampling devices and systems disclosed herein comprise at least one oligonucleotide primer, wherein the oligonucleotide primer has a sequence complementary to or corresponding to a Y chromosome sequence. In some embodiments, sampling devices and systems disclosed herein comprise a pair of oligonucleotide primers, wherein the pair of oligonucleotide primers have sequences complementary to or corresponding to a Y chromosome sequence. In some embodiments, sampling devices and systems disclosed herein comprise at least one oligonucleotide primer, wherein the oligonucleotide primer comprises a sequence complementary to or corresponding to a Y chromosome sequence. In some embodiments, sampling devices and systems disclosed herein comprise a pair of oligonucleotide primers, wherein the pair of oligonucleotide primers comprise sequences complementary to or corresponding to a Y chromosome sequence. In some embodiments, sampling devices and systems disclosed herein comprise at least one oligonucleotide primer, wherein the oligonucleotide primer consists of a sequence complementary to or corresponding to a Y chromosome sequence. In some embodiments, sampling devices and systems disclosed herein comprise a pair of oligonucleotide primers, wherein the pair of oligonucleotide primers consists of sequences complementary to or corresponding to a Y chromosome sequence. In some embodiments, the sequence(s) complementary to or corresponding to a Y chromosome sequence is at least 75% homologous to a wild-type human Y chromosome sequence. In some embodiments, the sequence(s) complementary to or corresponding to a Y chromosome sequence is at least 80% homologous to a wild-type human Y chromosome sequence. In some embodiments, the sequence(s) complementary to or corresponding to a Y chromosome sequence is at least 85% homologous to a wild-type human Y chromosome sequence. In some embodiments, the sequence(s) complementary to or corresponding to a Y chromosome sequence is at least 80% homologous to a wild-type human Y chromosome sequence. In some embodiments, the sequence(s) complementary to or corresponding to a Y chromosome sequence is at least 90% homologous to a wild-type human Y chromosome sequence. In some embodiments, the sequence(s) complementary to or corresponding to a Y chromosome sequence is at least 95% homologous to a wild-type human Y chromosome sequence. In some embodiments, the sequence(s) complementary to or corresponding to a Y chromosome sequence is at least 97% homologous to a wild-type human Y chromosome sequence. In some embodiments, the sequence(s) complementary to or corresponding to a Y chromosome sequence is 100% homologous to a wild-type human Y chromosome sequence. In some embodiments, sampling devices and systems disclosed herein are capable of tagging at least a portion of the cell-free nucleic acids (e.g., the amplified cfDNA). In some embodiments, the tagging comprises: (a) generating ligation competent cell-free DNA by one or more steps comprising: (i) generating a blunt end of the cell-free DNA, In some embodiments, a 5′ overhang or a 3′ recessed end is removed using one or more polymerase and one or more exonuclease; (ii) dephosphorylating the blunt end of the cell-free DNA; (iii) contacting the cell-free DNA with a crowding reagent thereby enhancing a reaction between the one or more polymerases, one or more exonucleases, and the cell-free DNA; or (iv) repairing or remove DNA damage in the cell-free DNA using a ligase; and (b) ligating the ligation competent cell-free DNA to adaptor oligonucleotides by contacting the ligation competent cell-free DNA to adaptor oligonucleotides in the presence of a ligase, crowding reagent, and/or a small molecule enhancer. In some embodiments, the methods further comprise pooling two or more biological samples, each sample obtained from a different subject. In some embodiments, the methods further comprise contacting the biological sample with a white blood cell stabilizer following obtaining the biological sample from the subject. In some embodiments, the one or more polymerases comprises T4 DNA polymerase or DNA polymerase I. In some embodiments, the one or more exonucleases comprises T4 polynucleotide kinase or exonuclease III. In some embodiments, the ligase comprises T3 DNA ligase, T4 DNA ligase, T7 DNA ligase, Taq Ligase, Ampligase, E. coli Ligase, or Sso7-ligase fusion protein. In some embodiments, the crowding reagent comprises polyethylene glycol (PEG), glycogen, or dextran, or a combination thereof. In some embodiments, the small molecule enhancer comprises dimethyl sulfoxide (DMSO), polysorbate 20, formamide, or a diol, or a combination thereof. In some embodiments, ligating in (b) comprises blunt end ligating, or single nucleotide overhang ligating. In some embodiments, the adaptor oligonucleotides comprise Y shaped adaptors, hairpin adaptors, stem loop adaptors, degradable adaptors, blocked self-ligating adaptors, or barcoded adaptors, or a combination thereof.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are biological entities containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed, such as a patient, or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

In general, the terms “cell-free polynucleotide,” “cell-free nucleic acid,” used interchangeably herein, refer to polynucleotides and nucleic acids that can be isolated from a sample without extracting the polynucleotide or nucleic acid from a cell. A cell-free nucleic acid may comprise DNA. A cell-free nucleic acid may comprise RNA. A cell-free nucleic acid is a nucleic acid that is not contained within a cell membrane, i.e., it is not encapsulated in a cellular compartment. In some embodiments, a cell-free nucleic acid is a nucleic acid that is not bounded by a cell membrane and is circulating or present in blood or other fluid. In some embodiments, the cell-free nucleic acid is cell-free before and/or upon collection of the biological sample containing it and is not released from the cell as a result of sample manipulation by man, intentional or otherwise, including manipulation upon or after collection of the sample. In some instances, cell-free nucleic acids are produced in a cell and released from the cell by physiological means, including, e.g., apoptosis, and non-apoptotic cell death, necrosis, autophagy, spontaneous release (e.g., of a DNA/RNA-lipoprotein complex), secretion, and/or mitotic catastrophe. In some embodiments, a cell-free nucleic acid comprises a nucleic acid that is released from a cell by a biological mechanism, (e.g., apoptosis, cell secretion, vesicular release). In further or additional embodiments, a cell-free nucleic acid is not a nucleic acid that has been extracted from a cell by human manipulation of the cell or sample processing (e.g., cell membrane disruption, lysis, vortex, shearing, etc.).

In some instances, the cell-free nucleic acid is a cell-free fetal nucleic acid. In general, the term, “cell-free fetal nucleic acid,” as used herein, refers to a cell-free nucleic acid, as described herein, wherein the cell-free nucleic acid is from a cell that comprises fetal DNA. In pregnant women, the cell-free DNA originating from the placenta can contribute a noticeable portion of the total amount of cell-free DNA. Placental DNA is often a good surrogate for the fetal DNA, because in most cases it is highly similar to the DNA of the fetus. Applications like chorionic villus sampling have exploited this fact to establish diagnostic application. Often, a large portion of cell-free fetal nucleic acids are found in maternal biological samples as a result of placental tissue being regularly shed during the pregnant subject's pregnancy. Often, many of the cells in the placental tissue shed are cells that contain fetal DNA. Cells shed from the placenta release fetal nucleic acids. Thus, in some instances, cell-free fetal nucleic acids disclosed herein are nucleic acids release from a placental cell.

As used herein, the term, “tag” generally refers to a molecule that can be used to identify, detect or isolate a nucleic acid of interest. The term, “tag,” may be used interchangeably with other terms, such as “label,” “adapter,” “oligo,” and “barcode,” unless specified otherwise. Note, however, that the term, “adapter,” can be used to ligate two ends of a nucleic acid or multiple nucleic acids without acting as a tag.

As used herein, the terms, “isolate,” “purify,” “remove,” “capture,” and “separate,” may all be used interchangeably unless specified otherwise.

As used herein, the terms, “clinic,” “clinical setting,” “laboratory” or “laboratory setting” refer to a hospital, a clinic, a pharmacy, a research institution, a pathology laboratory, a or other commercial business setting where trained personnel are employed to process and/or analyze biological and/or environmental samples. These terms are contrasted with point of care, a remote location, a home, a school, and otherwise non-business, non-institutional setting.

As used herein, the terms “homologous,” “homology,” or “percent homology” describe sequence similarity of a first amino acid sequence or a nucleic acid sequence relative to a second amino acid sequence or a nucleic acid sequence. In some instances, homology can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

Throughout the application, there is recitation of chromosome positions. These position numbers are in reference to Genome Build hg38 (UCSC) and GRCh38 (NCBI). A genome build may also be referred to in the art as a reference genome or reference assembly. It may be derived from multiple subjects. It is understood that there are multiple reference assemblies available and more reference assemblies may be produced over time. However, one skilled in the art would be able to determine the relative positions provided herein in another genome build or reference genome

The section headings used herein are for organizational purposes only and are not to be construed as limiting the subject matter described.

### EXAMPLES

The following examples are included for illustrative purposes only and are not intended to limit the scope of the invention.

### Example 1

### Sample Processing using a Device to Prepare a Sample for Analyte Detection

In an embodiment a sampling device comprises a materials processing module 910, a fluid routing network 950, and a fluid storage and actuation system 960. In an embodiment, fingerstick blood is added to a sample input 905 to be received by first processing module 912 of the materials processing module 910. The first processing module 912 comprises a filter. The blood is filtered in the first processing module 910 to produce blood plasma. A first valve 920 is activated and a pump of pump supply subsystem 962 withdraws the plasma from the first processing module 912 through a first junction 940 and into a reservoir of the fluid supply subsystem 962.

In an embodiment, the first fluid supply subsystem 962 also contains a mixture of magnetic beads and binding buffer which may be held in separate reservoirs or a single reservoir within the first fluid supply subsystem 962. The newly added plasma is mixed with this solution in the first fluid supply subsystem 962 for a period of time. In an embodiment, during mixing the cfDNA in the solution binds to the surfaces of the beads.

In an embodiment, after the mixing within the first fluid supply subsystem 962 is complete, the first valve 920 is deactivated (or closed) and second valve 922 is activated (or opened). The solution in the first fluid supply subsystem 962 is pumped up through a second fluid junction 942 and into the second processing module 914.

In an embodiment, after some time, the beads are immobilized in a channel by means of magnetophoresis. The liquid component of the solution is withdrawn back to a reservoir of the first fluid supply subsystem 962 as waste.

In an embodiment, after the waste solution is removed from the second processing module 914, the second valve 922 is deactivated and a third valve 932 is activated.

A second fluid supply subsystem 972 contains a wash solution that is pumped up into the second processing module 914 through the second fluid junction 942 and then withdrawn as a waste solution back into a reservoir of the second fluid supply subsystem. The third valve 932 is then deactivated and fourth valve 934 is activated.

In an embodiment, a third fluid supply subsystem 974 contains another wash solution that is pumped up into the second processing module 914 through the second junction 924 and then withdrawn as waste solution back into a reservoir of the third fluid supply subsystem 974. The fourth valve 934 is then deactivated and a fifth valve 924 is activated.

In an embodiment, a fourth fluid supply subsystem 964 contains an elution buffer that is pumped into the second processing module 914 through a third fluid junction 944 and the second fluid junction 942. After some time, the purified cfDNA desorbs from the immobilized beads into the buffer, becoming an eluate. The eluate is withdrawn from the second processing module 914 into the fourth fluid supply subsystem 964. The fifth valve 924 is deactivated and a seventh valve 936 is activated.

In an embodiment, a reaction activation buffer contained in a fifth fluid supply subsystem 976 is infused into a well in the third processing module 916 through a fourth fluid junction 946. The seventh valve 936 is deactivated and an eight valve 938 is activated.

In an embodiment, a master mix buffer contained in a sixth fluid supply subsystem 978 is infused into the same well holding the reaction activation buffer in the third processing module 916 through the fourth fluid junction 946. The eight valve 938 is then deactivated and a sixth valve 926 is activated.

In an embodiment, the eluate is then infused into the same well holding the reaction activation buffer and the master mix buffer in the third processing module 916 through the fourth fluid junction 946.

In an embodiment, the fourth fluid supply subsystem 964 actuates the mixing of solutions in the well in the third processing module 916 for some time.

In an embodiment, the third processing module 916 contains a heater that is turned on during the mixing process. The temperature of the solution climbs to a set point and is held there. This initiates an isothermal nucleic acid amplification reaction.

In an embodiment, after amplification, the sixth valve 926 is then deactivated and a ninth valve 928 is activated. The enriched DNA product is withdrawn from third processing module 916 through the fourth fluid junction 946 and a fifth fluid junction 948 into a reservoir of a seventh fluid supply subsystem 966.

In an embodiment, the seventh fluid supply subsystem 966 previously contains a dilution buffer. The enriched product is mixed with this buffer in seventh fluid supply subsystem 966. After missing, the ninth valve 928 is deactivated and tenth valve 930 is activated.

In an embodiment, a volume of diluted enriched product is pumped through the fifth fluid junction 948 and infused into a fourth processing module containing a chromatographic strip.

In an embodiment, a visual indication of a result develops in the fourth processing module 918 and is detected automatically by optical and electronic systems and processed as a detectable signal data output 985. While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device for preparing a sample for analyte detection comprising:
a processor comprising:
a filter configured to filter solid particulate from the sample, and
an enricher downstream from the filter and configured to increase a quantity of target analytes in the sample;
a fluid supply comprising a reagent and a pump that move the reagents from the fluid supply and move the sample to the enricher; and
a fluid routing network comprised of fluid pathway and valve to direct flow of the sample and the reagents to the enricher.

2. The device of claim 1, further comprising an electronics and software subsystem that controls the pump and the valve.

3. The device of claim 1, wherein in the valve is a rotary valve.

4. The device of claim 3, further comprising an electronics and software subsystem that controls the pump and the rotary valve.

5. The device of any one of claims 1-4, wherein the processor further comprises a washer downstream from the filter and upstream from the enricher and configured to separate the target analytes from other substances within the sample.

6. The device of any one of claims 1-4, wherein the processor further comprises a hybridizer downstream from the filter and upstream from the enricher, the hybridizer configured to bind the target analytes to one or more antibodies of high affinity.

7. The device of claim 6, wherein the processor further comprises an eluter downstream from the hybridizer and upstream from the enricher, the eluter configured to isolate analytes to be detected from the sample in an eluate.

8. The device of claim 7, wherein the processor further comprises a diluter downstream from the enricher, the diluter configured to dilute the eluate in an aqueous buffer.

9. The device of any one of claims 1-8, wherein the processor further comprises a detector downstream from all components of the processor, the detector configured to detect the target analytes in the eluate.

10. The device of claim 9, wherein the detector produces an optically detectable signal.

11. The device of claim 10, wherein the detector comprises a chromatography device.

12. The device of claim 11, wherein the chromatography device is a lateral flow assay.

13. The device of any one of claims 1-12, wherein at least one of the hybridizer, the eluter, the enricher, or the diluter comprise an air vent.

14. The device of any one of claims 1-7, wherein the processor further comprises a diluter downstream from the enricher, the diluter configured to dilute the sample in an aqueous buffer.

15. The device of any one of claims 1-8, wherein the processor further comprises a detector downstream from all components of the processor, the detector configured to detect the target analytes in the sample.

16. The device of any one of claims 1-15, wherein the sample has a volume comprising at most or about 400 microliters (μl), 350 μl, 300 μl, 250 μl, 200 μl, 150 μl, 100 μl, 50 μl, 45 μl, 40 μl, 35 μl, 30 μl, or less.

17. The device of any one of claims 1-16, wherein the sample is whole blood.

18. The device of any one of claims 1-17, wherein the target analytes comprise a target region of cell-free deoxyribonucleic acid (DNA).

19. The device of claim 18, wherein the cell-free DNA is fragmented.

20. The device of claim 18, wherein the sample comprises an amount of the target analytes comprising between or about 4pg to 100pg, 4pg to 150pg, 4pg to 200pg, 4pg to 250pg, 4pg to 300pg, 4pg to 350pg, 4pg to 400pg, 4pg to 450pg, 4pg to 500pg, 10pg to 100pg, 10pg to 150pg, 10pg to 200pg, 10pg to 250pg, 10pg to 300pg, 10pg to 350pg, 10pg to 400pg, 10pg to 450pg, 10pg to 500pg, 20pg to 100pg, 20pg to 150pg, 20pg to 200pg, 20pg to 250pg, 20pg to 300pg, 20pg to 350pg, 20pg to 400pg, 20pg to 450pg, 20pg to 500pg, 30pg to 100pg, 30pg to 150pg, 30pg to 200pg, 30pg to 250pg, 30pg to 300pg, 30pg to 350pg, 30pg to 400pg, 30pg to 450pg, or 30pg to 500pg.

21. A system for detecting molecular analytes comprising:
one or more devices of any one of claims 1 -20;
at least one controller for controlling the one or more devices;
and at least one interface for manipulating the at least one controller.

22. The system of claim 21, further comprising a sample collector configured to obtain the sample from a subject.

23. The system of claim 22, wherein the sample collector is operably coupled to a transdermal puncture device.

24. The system of claim 23, wherein the transdermal puncture device comprises a microneedle, microneedle array, or microneedle patch.

25. A method for preparing a sample and for analyte detection using the device of claim 9, the method comprising:
receiving the sample comprising the target analytes at an inlet of the filter;
filtering the sample with the filter, thereby producing a filtered sample;
mixing the filtered sample with the aqueous solution in the hybridizer;
hybridizing the filtered sample mixed with the aqueous solution in the hybridizer, thereby producing the hybridized solution;
mixing the hybridized solution with a solvent in the eluter, thereby producing the eluate;
mixing the eluate with an enrichment solution in the enricher;
enriching the eluate mixed with the enrichment solution in the enricher, thereby producing an enriched sample;
diluting the enriched sample with an aqueous buffer in the diluter, thereby producing a diluted sample;
introducing the diluted sample to the detector to create at least one optically detectable signal; and
producing an output data set from the at least one optically detectable signal.

26. The method of claim 25, wherein the aqueous solution comprises salts, polymer surfactants, buffers, and combinations thereof.

27. The method of claim 25, wherein the step of enriching comprises heating the enrichment solution mixed with the eluate.

28. The method of any one one of claims 25-27, wherein the detector comprises a chromatography device.

29. The method of claim 28, wherein the chromatography device is a lateral flow assay.

30. The method of any one of claims 25-29, wherein the sample has a volume comprising at most or about 400 microliters (μl), 350 μl, 300 μl. 250 μl, 200 μl, 150 μl, 100 μl, 50 μl, 45 μl, 40 μl, 35 μl, 30 μl, or less.

31. The method of any one of claims 25-30, wherein the sample comprises an amount of the target analytes comprising between or about 4pg to 100pg, 4pg to 150pg, 4pg to 200pg, 4pg to 250pg, 4pg to 300pg, 4pg to 350pg, 4pg to 400pg, 4pg to 450pg, 4pg to 500pg, 10pg to 100pg, 10pg to 150pg, 10pg to 200pg, 10pg to 250pg, 10pg to 300pg, 10pg to 350pg, 10pg to 400pg, 10pg to 450pg, 10pg to 500pg, 20pg to 100pg, 20pg to 150pg, 20pg to 200pg, 20pg to 250pg, 20pg to 300pg, 20pg to 350pg, 20pg to 400pg, 20pg to 450pg, 20pg to 500pg, 30pg to 100pg, 30pg to 150pg, 30pg to 200pg, 30pg to 250pg, 30pg to 300pg, 30pg to 350pg, 30pg to 400pg, 30pg to 450pg, or 30pg to 500pg.

32. The method of any one of claims 25-30, wherein the cfDNA is fragmented.

33. A method for detection of cell-free DNA (cfDNA) in blood, the method comprising:
receiving a sample comprising whole blood;
filtering the sample to substantially remove solid particles, the solid particles comprising red blood cells, white blood cells, apoptotic bodies, viral particles, or combinations thereof, thereby producing blood plasma;
mixing the blood plasma with a first aqueous solution;
binding cell-free DNA (cfDNA) molecules to a surface of one or more paramagnetic microspheres;
separating the microspheres from the solution of the blood plasma and the first aqueous solution;
washing the microspheres with a second aqueous solution;
separating the microspheres from the cfDNA molecules using an elution, thereby producing an eluate comprising purified cfDNA molecules;
enriching the eluate to increase a number of the cfDNA molecules;
diluting the eluate with an aqueous buffer;
introducing the eluate to a chromatographic paper strip thereby producing one or more optically detectable signals; and
outputting a dataset comprising detection of the one or more optically detectable signals.

34. A method for detection of quantities of target antigens in blood, the method comprising:
receiving a sample comprising whole blood;
filtering the sample to substantially remove solid particles, the solid particles comprising red blood cells, white blood cells, apoptotic bodies, or, viral particles, or combinations thereof, thereby producing blood plasma;
mixing the blood plasma with a first aqueous solution;
binding the target antigens to DNA labeled antibodies;
binding the target antigens to primary antibodies coated on more microspheres, wherein the primary antibodies selectively bind to the target antigens thereby producing bound triads comprising a target antigens, a primary antibodies, and a DNA labeled antibodies;
washing the bound triads and the microspheres with a second aqueous solution;
removing the bound triads from the microspheres using an elution;
enriching a solution containing the bound triads to produce an enriched solution;
diluting the enriched solution with an aqueous buffer;
introducing the enriched solution to a chromatographic paper strip thereby producing one or more optically detectable signals; and
outputting a dataset comprising detection of the one or more optically detectable signals,
wherein a quantity of the one or more optically detectable signals is proportional to a quantity of the target antigens in the enriched solution.

35. The method of claim 33 or 34, wherein the sample has a volume comprising at most or about 400 microliters (μl), 350 μl, 300 μl. 250 μl, 200 μl, 150 μl, 100 μl, 50 μl, 45 μl, 40 μl, 35 μl, 30μl or less.

36. The method of any one of claims 33-35, wherein the sample comprises an amount of the cfDNA comprising between or about 4pg to 100pg, 4pg to 150pg, 4pg to 200pg, 4pg to 250pg, 4pg to 300pg, 4pg to 350pg, 4pg to 400pg, 4pg to 450pg, 4pg to 500pg, 10pg to 100pg, 10pg to 150pg, 10pg to 200pg, 10pg to 250pg, 10pg to 300pg, 10pg to 350pg, 10pg to 400pg, 10pg to 450pg, 10pg to 500pg, 20pg to 100pg, 20pg to 150pg, 20pg to 200pg, 20pg to 250pg, 20pg to 300pg, 20pg to 350pg, 20pg to 400pg, 20pg to 450pg, 20pg to 500pg, 30pg to 100pg, 30pg to 150pg, 30pg to 200pg, 30pg to 250pg, 30pg to 300pg, 30pg to 350pg, 30pg to 400pg, 30pg to 450pg, or 30pg to 500pg.

37. The method of claim 36, wherein the cfDNA is fragmented.

38. A method for analyzing a quantity of target antigens in a sample, the method comprising:
removing solid particles from a sample using a filter, thereby producing a filtered sample;
mixing the filtered sample with a first aqueous solution;
contacting DNA labeled antibodies and primary antibodies attached to microspheres to the sample, wherein the sample comprises target antigens;
binding the target antigens in the sample to the DNA labeled antibodies and the primary antibodies, thereby producing a conjugate solution comprising bound triads of a target antigen, a primary antibody, and a DNA labeled antibody, wherein the bound triad is attached to a microsphere;
washing the conjugate solution with a second aqueous solution;
removing the DNA labeled antibodies from the bound triads using an elution;
enriching the one or more DNA labeled antibodies, thereby producing an enriched solution;
diluting the enriched solution with an aqueous buffer;
introducing the enriched solution to a chromatographic paper strip, thereby producing detectable signals; and
outputting a dataset comprising detection of the detectable signals,
wherein a quantity of the detectable signals is proportional to the quantity of the target antigens.

39. The method of claim 38, wherein the sample is whole blood.

40. The method of claim 38 or 39, wherein the sample has a volume comprising at most or about 400 microliters (μl), 350 μl, 300 μl. 250 μl, 200 μl, 150 μl, 100 μl, 50 μl, 45 μl, 40 μl, 35 μl, 30 μl or less.

41. The method of any one of claims 38-40, wherein removing the solid particles comprises removing red blood cells, white blood cells, apoptotic bodies, or viral particles, or combinations thereof, from the sample, thereby producing blood plasma.

42. A device for preparing a sample for analyte detection, the device comprising:
a processor comprising
a filter comprising a filter inlet to receive the sample and a filter outlet to output a filtered sample: and
an enricher configured to increase a number of target analytes for detection;
a fluid routing network comprising:
a first fluid pathway coupling the filter outlet to the enricher;
a first valve along the first fluid pathway;
a second valve along the first fluid pathway; and
a first fluid junction positioned between the first and second valves and coupling a first pump channel to the first fluid pathway;
a fluid supply comprising:
a first pump in fluid communication with the first pump channel and a first reservoir containing an aqueous solution, wherein the first pump is configured to supply the aqueous solution to the enricher and transport the filtered sample through the first fluid pathway; and
an electronics and software subsystem that controls the first pump, the first valve, and the second valve.

43. The device of claim 42, wherein the aqueous solution and the filtered sample are mixed in the enricher to form a sample solution, and wherein the sample solution is heated within the enricher to produce an enriched sample.

44. The device of claim 43, wherein the enriched sample is output from the device by the first pump through an enricher outlet.

45. The device of claim 42 or 43, wherein the aqueous solution comprises salts, polymer surfactants, buffers, or a combination thereof.

46. The device of any one of claims 42-45, wherein the processor further comprises a detector comprising a chromatography device.

47. The device of claim 46, wherein the fluid routing network further comprises:
a second fluid pathway coupling an outlet of the enricher to an inlet of the detector;
a third valve along the second fluid pathway;
a fourth valve along the second fluid pathway; and
a second fluid junction in fluid communication with the second fluid pathway and positioned between the third and fourth valves.

48. The device of claim 47, wherein the fluid supply further comprises:
a second pump in fluid communication with the second fluid junction and a second reservoir containing an aqueous buffer, wherein the second pump is configured to supply the aqueous buffer to the second fluid junction;
wherein:
the electronics and software subsystem further controls the third valve, the fourth valve, and the second pump,
the enriched sample and the aqueous buffer are mixed within the second fluid junction to produce a buffered sample, and
the second pump transports the buffered sample to the inlet of the detector.

49. A device for preparing a sample for analyte detection comprising:
a processor comprising:
a filter comprising a filter inlet to receive the sample and a filter outlet to output a filtered sample;
a hybridizer configured to receive the filtered sample and hybridize the filtered sample to produce a hybridized sample;
an eluter configured to receive the hybridized sample and elute the hybridized sample to produce an eluate; and
an enricher configured to increase a number of analytes in the eluate for detection, thereby producing an enriched sample;
a fluid routing network comprising:
a first fluid pathway coupling the filter outlet to the enricher;
a first valve along the first fluid pathway;
a second valve along the first fluid pathway;
a first fluid junction positioned between the first and second valves and coupling a first pump channel to the first fluid pathway;
a third valve along the first fluid pathway;
a second fluid junction provided between the second and third valves, the second fluid junction coupling the first fluid pathway, a second pump channel, a third pump channel, and the hybridizer;
a fourth valve along the first fluid pathway;
a third fluid junction in fluid provided between the third and fourth valves and coupling the first fluid pathway with a fourth pump channel;
a fifth valve along the first fluid pathway;
a fourth fluid junction provided between the fourth and fifth valves and coupling the eluter to the first fluid pathway;
a sixth valve along the first fluid pathway;
a fifth fluid junction in provided between the fifth and sixth valves and coupling a fifth pump channel to the first fluid pathway; and
a sixth fluid junction coupling the enricher and the first fluid pathway,
wherein the fluid supply comprises:
a first pump in fluid communication the first pump channel and a first reservoir containing an aqueous solution, wherein the first pump is configured to supply the aqueous solution to the hybridizer and transport the filtered sample through the first fluid pathway;
a second pump in fluid communication with the second pump channel and a second reservoir containing a washing solution, wherein the second pump is supplies the washing solution to the hybridizer;
a third pump in fluid communication with the third pump channel and a third reservoir, wherein the third pump is configured to remove the washing solution from the hybridizer and deposit the washing solution into the third reservoir;
a fourth pump in fluid communication with the fourth pump channel and a fourth reservoir containing a solvent, wherein the fourth pump is configured to supply the solvent to the eluter and transport the hybridized sample through the first fluid pathway; and
a fifth pump in fluid communication with the fifth pump channel and a fifth reservoir containing an enrichment solution, wherein the fifth pump is configured to supply the enrichment solution to the enricher and transport the eluate through the first fluid pathway.

50. The device of claim 49, wherein the processor further comprises a diluter to dilute the enriched sample and produces a diluted sample; and wherein the fluid routing network further comprises:
a second fluid pathway coupling the enricher to the diluter;
a seventh valve along the second fluid pathway;
an eighth valve along the second fluid pathway;
a seventh fluid junction in provided between the seventh and eighth valves and coupling a sixth pump channel to the second fluid pathway; and
an eighth fluid junction coupling the diluter to the second fluid pathway; and
wherein the fluid supply further comprises a sixth pump in fluid communication with the sixth pump channel and a sixth reservoir containing an aqueous buffer, wherein the sixth pump is configured to supply the aqueous buffer to the diluter and transport the enriched sample through the second fluid pathway.

51. The device of claim 50, wherein the processor further comprises a detector;
wherein the fluid routing network further comprises:
a third fluid pathway coupling the diluter to the detector;
a ninth valve along the third fluid pathway;
a tenth valve along the third fluid pathway; and
a ninth fluid junction in provided between the ninth valves and tenth valves and coupling a seventh pump channel to the third fluid pathway; and
wherein the pump supply further comprises a seventh pump in fluid communication the seventh pump channel, wherein the seventh pump is configured to transport the diluted sample through the third fluid pathway.
